(19) United States
(12) Patent Application Publication
(10) Pub. No.: US 2026/0014120 A1
(43) Pub. Date: Jan. 15, 2026

(54) COMPOSITIONS AND METHODS FOR TOPICAL DELIVERY OF SALTS OF CARNOSINE AND CARNITINE

(71) Applicant: NeuviDerm LLC, Pinecrest, FL (US)

(72) Inventors: Jacob J. Miguel, Pinecrest, FL (US); Spencer Bouhadir, Lake Worth, FL (US)

(21) Appl. No.: 18/768,947

(22) Filed: Jul. 10, 2024

(51) Int. Cl. A61K 31/4164; A61K 9/08; A61K 31/205; A61K 31/4166; A61K 31/60; A61K 31/728; A61K 47/12; A61K 47/18; A61K 47/40

(52) U.S. Cl. CPC A61K 31/4164; A61K 9/08; A61K 31/205; A61K 31/4166; A61K 31/60; A61K 31/728; A61K 47/12; A61K 47/183; A61K 47/40

(57) ABSTRACT

Provided herein is, inter alia, a hydrogel composition and methods for the treatment of skin conditions such as eczema, dandruff, and acne. The compositions comprise a synergistic blend of active ingredients including salts of carnosine, salts of carnitine, and cyclodextrin, which are formulated to be maintained in solution. The compositions disclosed herein are capable of altering gene expression related to skin health and can be manufactured through a method that involves forming carnosine and/or carnitine salts and combining them with cyclodextrin in solution.

### BACKGROUND OF THE INVENTION

Eczema, dandruff, and acne are common dermatological conditions related to inflammation of the skin that affect a large portion of the population worldwide. These conditions are not life-threatening but can cause considerable discomfort and can have a psychological impact due to their visibility.

Eczema, also known as atopic dermatitis, is characterized by red, inflamed, and itchy skin. The condition is often chronic and can be triggered by various factors including allergens, stress, and irritants. Current treatments for eczema include topical corticosteroids, moisturizers, and immunomodulators. However, these treatments may have side effects such as skin thinning and resistance to therapy.

Dandruff is an inflammatory scalp condition that is marked by flaking and sometimes mild itchiness. It can be caused by a number of factors, including dry skin, sensitivity to hair products, and the growth of a specific type of fungus on the scalp. Treatment typically involves the use of anti-dandruff shampoos containing active ingredients such as zinc pyrithione, selenium sulfide, and ketoconazole. While these treatments can be effective, they may not provide a long-term solution and can lead to scalp irritation with prolonged use.

Acne is a skin condition that occurs when hair follicles become clogged with oil and dead skin cells, leading to whiteheads, blackheads, or pimples and accompanying inflammation. Acne is particularly common among teenagers but can affect people of all ages. Treatments for acne include over-the-counter gels, creams, and soaps, as well as prescription medications like antibiotics and retinoids. These treatments can be effective but may cause side effects such as dryness, redness, and irritation.

There is a continuous search for new treatment formulations that can effectively manage these inflammatory skin conditions with fewer side effects. Compositions that can treat skin conditions by topical application, such as hydrogels, are particularly desirable.

Hydrogels, which are three-dimensional polymer networks capable of holding large amounts of water, offer a promising delivery system for topical applications due to their biocompatibility and ability to provide sustained release of active ingredients. Preparing hydrogel formulations comprising certain anti-inflammatory agents, however, can be challenging. The incorporation of carnosine/carnitine salts into hydrogel matrices can have unpredictable interactions between the peptide and the polymer components. These interactions can alter the gel structure, affecting the mechanical properties and the release profile of the hydrogel. For example, carnosine, a dipeptide composed of beta-alanine and L-histidine, has been recognized for its antioxidant, anti-glycation, and potential wound-healing properties. Carnitine, another anti-inflammatory compound, is a quaternary ammonium compound derived from the amino acids lysine and methionine. However, the incorporation of carnosine/carnitine or their salts into hydrogel systems for topical treatment of inflammatory skin conditions is not straightforward due to physicochemical properties and stability concerns.

One of the primary difficulties in formulating carnosine salts within hydrogels is the peptide's inherent instability in aqueous solutions. Carnosine can undergo hydrolysis, particularly in the presence of carnosinase enzymes found in human skin, which can rapidly degrade carnosine, reducing its therapeutic effectiveness. Additionally, the pH of the formulation can affect the stability of carnosine salts, necessitating careful pH adjustments and buffering to maintain the integrity of the active ingredient. Furthermore, carnitine salts, particularly the biologically active L-carnitine, are hygroscopic and can attract and hold water molecules, which may affect the stability and consistency of hydrogel formulations. This hygroscopic nature can lead to issues with the shelf-life and texture of the final product, as the hydrogel may become too wet or degrade over time due to the presence of excess moisture.

Another challenge is ensuring the homogenous dispersion of carnosine/carnitine salts throughout the hydrogel matrix. Incompatibility with the gel constituents can lead to phase separation, resulting in an uneven distribution of the active ingredient, which can compromise the efficacy and reproducibility of the final product. Furthermore, the development of combination therapies that include carnosine/carnitine salts in hydrogels often requires the addition of other active ingredients to address specific skin conditions. The compatibility of carnosine/carnitine with these additional components, as well as the overall stability of the formulation, requires extensive research and optimization.

Overall, the development of stable and effective carnosine/carnitine salt-hydrogel combinations for therapeutic applications is a complex process that requires a deep understanding of peptide chemistry, polymer science, and formulation strategies to overcome the inherent challenges associated with these components. There exists a need for new formulations of carnosine/carnitine salts with a hydrogel to overcome issues with solubility, complexing, chemical stability, and chemical compatibility that are effective for use with topical treatment of inflammatory skin conditions. The present disclosure addresses these and other needs.

### SUMMARY OF THE DISCLOSURE

According to some aspects, the present disclosure provides compositions and formulations comprising salts of carnosine and carnitine in a hydrogel for topical delivery. In some embodiments, the combinations of salts are effective to achieve solubility and chemical compatibility with polymers. In some embodiments, the formulations disclosed herein comprise oligosaccharides (e.g., cyclodextrin) with polymers (e.g., hyaluronic acid of various molecular weights) that surprisingly do not form insoluble complexes. In some embodiments, this maintenance of solubility achieved via the combination of components of the formulation and/or the order of combining the components of the formulation. In some embodiments, salts of carnosine and carnitine are formed by reaction with an acid in isolation, and prior to oligosaccharide and/or polymer addition. In some embodiments, the combination of components is effective to solubilize otherwise hydrophobic compositions. For example, allantoin is insoluble in aqueous solutions and gels, but as disclosed herein combining the carnosine/carnitine salts with a polymer, osmolyte, cyclodextrin blend can solubilize allantoin without recrystallization occurring.

According to some aspects, the present disclosure provides a composition for treating inflammatory skin conditions, such as eczema, dandruff, and acne. In some embodiments, the composition comprises carnosine, lactic acid, cyclodextrin, carnitine, trimethylglycine, and sodium hyaluronate. The present disclosure also provides the types and ranges of these components, and also includes additional ingredients such as salicylic acid, glycolic acid, and allantoin.

According to some aspects, the present disclosure provides composition that is effective to treat eczema in a subject in need thereof comprising a solution comprising carnosine, lactic acid, cyclodextrin, carnitine, trimethylglycine, and sodium hyaluronate. In some embodiments, a composition for treatment of eczema comprises carnosine, lactic acid, cyclodextrin, carnitine, trimethylglycine, and sodium hyaluronate, wherein the carnosine, carnitine, lactic acid, cyclodextrin and sodium hyaluronate are present in an amount that is effective to maintain the carnosine, carnitine, lactic acid, cyclodextrin and sodium hyaluronate is in solution, and wherein the composition is effective to treat eczema. In some embodiments, the composition further comprises one or more of allantoin and glycolic acid. In some embodiments, the carnosine and lactic acid form a carnosine lactate salt and the carnitine and lactic acid form a carnitine lactate salt. In some embodiments, the carnosine and glycolic acid form a carnosine glycolate salt and the carnitine and glycolic acid form a carnitine glycolate salt. In some embodiments, the ratio of salt to cyclodextrin is between 1:1 to 3:1 by weight. In some embodiments, the carnosine comprises L-carnosine, D-carnosine, acetyl-carnosine, derivatives thereof, and combinations thereof. In some embodiments, the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof. In some embodiments, the cyclodextrin is non-crosslinked.

In some embodiments, the composition comprises 0.5-6% carnosine; 0.3-5% lactic acid; 0.1-1% hydroxypropyl gamma cyclodextrin; 0.5-3% carnitine; 3-10% trimethylglycine; and 0.1-0.8% sodium hyaluronate. In some embodiments, the composition comprises one or more of 0.4-3.5% salicylic acid, 0.2-3% glycolic acid, and 0.5% allantoin. In some embodiments, the composition comprises 2% carnosine; 1.4% lactic acid; 0.2% hydroxypropyl gamma cyclodextrin; 1% carnitine; 7% trimethylglycine; 0.2% sodium hyaluronate; and 0.5% allantoin. In some embodiments, the composition comprises 5% carnosine; 2.8% lactic acid; 0.3% glycolic acid; 0.5% hydroxypropyl gamma cyclodextrin; 2% carnitine; 7% trimethylglycine; 0.6% sodium hyaluronate; and 0.5% allantoin. In some embodiments, the composition comprises 2% carnosine; 1.4% lactic acid; 0.2% hydroxypropyl gamma cyclodextrin; 1% carnitine; 7% trimethylglycine; 0.2% sodium hyaluronate; 0.5% allantoin; 0.5% glycerin; 8% 1,3-propanediol; 2% pentylene glycol; 0.6% phenoxyethanol; 0.05% ethylhexylglycerine; and 0.5% allantoin. In some embodiments, the composition comprises 5% carnosine; 2.8% lactic acid; 0.3% glycolic acid; 0.5% hydroxypropyl gamma cyclodextrin; 2% carnitine; 7% trimethylglycine; 0.6% sodium hyaluronate; 0.5% glycerin; 8% 1,3-propanediol; 2% pentylene glycol; 0.6% phenoxyethanol; 0.05% ethylhexylglycerine; and 0.5% allantoin. In some embodiments, the composition comprises at least one pharmaceutically acceptable carrier or excipient. In some embodiments, the composition is a patch, a cream, an ointment, a powder, an aerosol spray, or a hydrogel. In some embodiments, the composition is a hydrogel.

According to some aspects, the present disclosure provides a method of treating eczema in a subject in need thereof comprising administering to the subject an effective amount of the composition disclosed herein. In some embodiments, the composition is administered topically. In some embodiments, the composition is administered every hour, every two hours, every three hours, every six hours, every twelve hours, every day, every two days, every three days, every five days, every seven days, every ten days, or every fourteen days.

According to some aspects, the present disclosure provides a composition that is effective to treat dandruff in a subject in need thereof comprising a solution comprising carnosine; salicylic acid; lactic acid; glycolic acid, cyclodextrin; carnitine; trimethylglycine; and sodium hyaluronate. In some embodiments a composition for treatment of dandruff comprises carnosine; salicylic acid; lactic acid; glycolic acid, cyclodextrin; carnitine; trimethylglycine; and sodium hyaluronate; wherein the carnosine, carnitine, lactic acid, salicylic acid, cyclodextrin and sodium hyaluronate are present in an amount that is effective to maintain the carnosine, carnitine, lactic acid, salicylic acid, cyclodextrin and sodium hyaluronate in solution; and wherein the composition is effective to treat dandruff. In some embodiments, one or more of the carnosine and carnitine react with one or more of salicylic acid, lactic acid, glycolic acid to form a salt. In some embodiments, the salt is one or more of carnosine salicylate and carnitine glycolate. In some embodiments, the carnosine and glycolic acid form a carnosine glycolate salt and the carnitine and glycolic acid form a carnitine glycolate salt. In some embodiments, the ratio of salt to cyclodextrin is between 1:1 to 1:3 by weight. In some embodiments, the ratio of salt to cyclodextrin is between 1:1 to 1:3 by weight. In some embodiments, the ratio of salt to cyclodextrin is between 1:1 to 3:1 by weight. In some embodiments, the ratio of salt to cyclodextrin is between 1:1 to 3:1 by weight. In some embodiments, the ratio of salt to cyclodextrin is between 1:1 to 3:1 by weight. In some embodiments, the ratio of salt to cyclodextrin is between 1:1 to 3:1 by weight. In some embodiments, the carnosine comprises L-carnosine, D-carnosine, acetyl-carnosine, derivatives thereof, and combinations thereof. In some embodiments, the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof. In some embodiments, the cyclodextrin is non-crosslinked.

In some embodiments, the composition comprises 0.5-6% carnosine; 0.4-3.5% salicylic acid; 0.3-5% lactic acid; 0.2-3% glycolic acid, 0.1-1% cyclodextrin; 0.5-3% carnitine; 3-10% trimethylglycine; and 0.1-0.8% sodium hyaluronate. In some embodiments, the composition comprises 4% carnosine; 1.8% salicylic acid; 0.8% lactic acid; 0.5% glycolic acid, 0.5% cyclodextrin; 1% carnitine; 0.5% glycerin; 8% 1,3-propanediol; 2% pentylene glycol; 3% trimethylglycine; 0.2% sodium hyaluronate; 0.6% phenoxyethanol; 0.05% ethylhexylglycerin. In some embodiments, the composition comprises at least one pharmaceutically acceptable carrier or excipient. In some embodiments, the composition is a patch, a cream, an ointment, a powder, an aerosol spray, or a hydrogel. In some embodiments, the composition is a hydrogel.

According to some aspects, the present disclosure provides a method of treating dandruff in a subject in need thereof comprising administering to the subject an effective amount of the composition disclosed herein. In some embodiments, the composition is administered topically. In some embodiments, the composition is administered every hour, every two hours, every three hours, every six hours, every twelve hours, every day, every two days, every three days, every five days, every seven days, every ten days, or every fourteen days.

According to some aspects, the present disclosure provides a composition that is effective to treat acne comprising a solution comprising carnosine; salicylic acid; lactic acid; glycolic acid, cyclodextrin; carnitine; trimethylglycine; and sodium hyaluronate. In some embodiments, a composition for treatment of acne comprises carnosine; salicylic acid; lactic acid; glycolic acid, cyclodextrin; carnitine; trimethylglycine; and sodium hyaluronate; wherein the carnosine, carnitine, lactic acid, salicylic acid, cyclodextrin and sodium hyaluronate are present in an amount that is effective to maintain the carnosine, carnitine, lactic acid, salicylic acid, cyclodextrin and sodium hyaluronate in solution; and wherein the composition is effective to treat acne. In some embodiments, one or more of the carnosine and carnitine react with one or more of salicylic acid, lactic acid, glycolic acid to form a salt. In some embodiments, the salt is one or more of carnosine salicylate and carnitine glycolate. In some embodiments, the carnosine and glycolic acid form a carnosine glycolate salt and the carnitine and glycolic acid form a carnitine glycolate salt. In some embodiments, the ratio of salt to cyclodextrin is between 1:1 to 3:1 by weight. In some embodiments, the carnosine comprises L-carnosine, D-carnosine, acetyl-carnosine, derivatives thereof, and combinations thereof. In some embodiments, the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof. In some embodiments, the cyclodextrin is non-crosslinked.

In some embodiments, the composition comprises 0.5-6% carnosine; 0.4-3.5% salicylic acid; 0.3-5% lactic acid; 0.2-3% glycolic acid, 0.1-1% cyclodextrin; 0.5-3% carnitine; 3-10% trimethylglycine; and 0.1-0.8% sodium hyaluronate. In some embodiments, the composition comprises 0.05% EDTA, 2% carnosine, 0.6% Salicylic acid, 0.45% lactic acid, 0.5% glycerin, 0.5% gamma-cyclodextrin, 2% pentylene glycol, 8% propanediol, 0.7% phenoxyethanol, 0.1% ethylhexylglycerin, and 0.6% hyaluronic acid. In some embodiments, the composition comprises 0.05% EDTA, 2% carnosine, 0.6% Salicylic acid, 0.45% lactic acid, 1% carnitine, 0.44% glycolic acid, 0.5% glycerin, 0.5% gamma-cyclodextrin, 2% pentylene glycol, 8% propanediol, 0.7% phenoxyethanol, 0.05% ethylhexylglycerin, and 0.6% hyaluronic acid. In some embodiments, the composition comprises 2% carnosine, 1.8% Salicylic acid, 0.8% lactic acid, 1% carnitine, 0.5% glycolic acid, 0.5% hydroxypropyl gamma-cyclodextrin, 8% propanediol, 2% pentylene glycol, 3% trimethylglycine, 0.2% sodium hyaluronate, 0.6% phenoxyethanol, and 0.05% ethylhexylglycerin. In some embodiments, the composition comprises at least one pharmaceutically acceptable carrier or excipient. In some embodiments, the composition is a patch, a cream, an ointment, a powder, an aerosol spray, or a hydrogel. In some embodiments, the composition is a hydrogel.

According to some aspects, the present disclosure provides, a method of treating acne in a subject in need thereof comprising administering to the subject an effective amount of the composition as disclosed herein. In some embodiments, the composition is administered topically. In some embodiments the composition is administered every hour, every two hours, every three hours, every six hours, every twelve hours, every day, every two days, every three days, every five days, every seven days, every ten days, or every fourteen days.

According to some aspects, the present disclosure provides a method for altering gene expression of a human skin cell in a human subject comprising contacting the human skin cell with a composition according to any embodiment disclosed herein; wherein the gene expression is one or more of the genes hFLG, hFGF-2, hEGF-1, hCOL1A1, hCOL1A2, hELN, hIGF-1, hTGF-β2, hMMP-2, hFGF-7, and hSDF-1. In some embodiments, one or more of the genes hFLG, hFGF-2, hEGF-1, hCOL1A1, hCOL1A2, hELN, hIGF-1, and hTGF-β2 are upregulated. In some embodiments, one or more of the genes hMMP-2, hFGF-7, and hSDF-1 are downregulated.

According to some aspects, the present disclosure provides a method of making a hydrogel salt composition comprising the steps of: contacting one or more of carnosine and carnitine with an acid to form a carnosine and/or carnitine salt; and combining the carnosine and/or carnitine salt with a cyclodextrin, wherein the ratio of carnosine and/or carnitine salt to cyclodextrin is between 1:1 and 3:1 by weight and wherein the carnosine and/or carnitine salt and cyclodextrin stay in solution. In some embodiments, the acid is one or more of lactic acid, salicylic acid, and glycolic acid. In some embodiments, the carnosine and/or carnitine salt is one or more of carnosine lactate, carnosine salicylate, carnosine glycolate, carnitine lactate, carnitine salicylate, and carnitine glycolate. In some embodiments, the cyclodextrin is alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof. In some embodiments, the carnosine comprises L-carnosine, D-carnosine, acetyl-carnosine, derivatives thereof, and combinations thereof. In some embodiments, the method further comprises the step of adding allantoin to the solution of carnosine and/or carnitine salt and cyclodextrin. In some embodiments, the method further comprises the step of adding trimethyglycine 1,3-propanediol, glycerin, and phenoxyethanol to the solution of carnosine and/or carnitine salt, cyclodextrin and allantoin. In some embodiments, the method further comprises the step of adding one or more of hyaluronic acid and sodium hyaluronate to the solution of carnosine and/or carnitine salt, cyclodextrin, allantoin, trimethyglycine 1,3-propanediol, glycerin, and phenoxyethanol. In some embodiments, the hyaluronic acid has a low, medium, or high molecular weight. In some embodiments, the one or more of hyaluronic acid and sodium hyaluronate is added last to prevent insoluble complexes from forming.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows the gene expression changes of human skin cells following treatment with carnosine, which is the main active ingredient of the composition according to certain embodiments disclosed herein.

### DETAILED DESCRIPTION

### Definitions

The term “about” is used here in conjunction with numeric values to include normal variations in measurements as expected by persons skilled in the art, and is understood have the same meaning as “approximately” and to cover a typical margin of error, such as +5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination y two or more of the listed elements.

Any amounts (e.g., concentrations) of components in a composition given as a percentage (%) refer to a percentage by weight per volume unless otherwise indicated.

As used herein, a “humectant” refers to a substance having an affinity for water and which provides stabilizing action on the water content of a material.

As used herein, the term “nonionic surfactant” refers to a molecule that acts as an uncharged surfactant. Surfactants are chemical compounds that decrease the surface tension or interfacial tension between two liquids, a liquid and a gas, or a liquid and a solid.

As used herein the term “preservative” refers to any known pharmaceutically acceptable preservative that functions by inhibiting bacteria, fungi, yeast, mold, other microbe. Suitable preservatives include but are not limited to antimicrobial agents. In some embodiments, antimicrobial agents comprise sodium benzoate, paraben, benzyl alcohol, sorbic acid, triclosan, phenoxyisopropanol, diazolidinyl urea, bronopol, Alkyl (C12-22) trimethyl ammonium bromide, Alkyl (C12-22) trimethyl ammonium chloride, Benzalkonium chloride, Benzalkonium bromide, Benzalkonium saccharinate, ethylhexylglycerin, phenoxyethanol, or a combination thereof. In some embodiments, the preservatives include one or more of parabens, alkyl parabens, aromatic alcohols, glycols, and chelating agents (e.g., EDTA, sodium phytate).

As used herein, “subject” refers to any mammal, including humans, domestic and farm animals, and zoo, sports, or pet animals, such as dogs, horses, cats, sheep, pigs, cows, etc. The preferred subject herein is a human subject, including adults, children, and the elderly.

The term “topical” application refers to application to skin, dermis or tissue site, and application to such tissue sites may include application adjacent to or within the tissue site.

The terms “treat,” “treated,” or “treating” as used herein refers to therapeutic treatment, cosmetic treatment and/or prophylactic or preventative measures, wherein the object is to prevent or slow down (lessen) an undesired physiological condition, disorder or disease, or to obtain beneficial or desired clinical results. As used herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

As used herein, the term a “therapeutically effective amount” is an amount sufficient to effect beneficial or desired results. A therapeutically effective amount can be administered in one or more doses. The therapeutically effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the composition being administered.

### Compositions

The compositions of the present disclosure can be used to treat any disorder, disease, or condition that would benefit from an agent that reduces inflammation in the skin. In some embodiments, the compositions of the present disclosure can be used to treat eczema, dandruff, and/or acne.

In some aspects, the present disclosure provides a composition comprising a delivery system that is effective to penetrate human skin. In some embodiments, the delivery system comprises a penetration-enhancing agent and/or surfactant ingredient or combinations thereof. In certain embodiments, the delivery system is effective to transport one or more active ingredients into the epidermis or dermis. In some aspects, the composition of the present disclosure is in the form of a hydrogel. As used herein, a “hydrogel” means a composition that comprises macromolecular compounds characterized by high water absorption capacity. In some embodiments, the macromolecular compounds of the hydrogel comprise cross-linked networks of macromolecular compounds.

### Eczema Formulations

According to some aspects, the present disclosure provides a composition for treatment of the inflammatory skin condition eczema, comprising one or more of carnosine, lactic acid, cyclodextrin, carnitine, trimethylglycine, and sodium hyaluronate. In some embodiments, the carnosine and/or carnitine form a salt via reaction with an acid. In some embodiments, the composition further comprises one or more of salicylic acid and glycolic acid.

In some embodiments, the carnosine comprises L-carnosine, D-carnosine, acetyl-carnosine, a derivative thereof, or a combination thereof. In some embodiments, the carnosine comprises L-carnosine. In some embodiments, the carnitine comprises L-carnitine, D-carnitine, acetyl-carnitine, propionyl-L-carnitine, a derivative thereof, or a combination thereof. In some embodiments, the compositions for treating eczema comprises, by weight of the total composition, 0.1%-10% (0.2, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnosine. In some embodiments, the compositions for treating eczema comprises, by weight of the total composition, 0.5%-10% (0.1%-10% (0.2, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnitine.

In some embodiments, the compositions for treating eczema comprises one or more of carnosine, carnitine, and cyclodextrin. In some embodiments, the compositions for treating eczema comprises L-carnosine and gamma cyclodextrin.

In some embodiments, the composition comprises a cyclic oligosaccharide-based polymer, such as an alpha cyclodextrin, beta cyclodextrin, a gamma cyclodextrin, hydroxypropyl gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glocosyl-β-cyclodextrin, maltosyl-β-cyclodextrin or any derivative thereof, and any combination thereof. In some embodiments, the cyclic oligosaccharide-based polymer is non-crosslinked. In some embodiments, the cyclic oligosaccharide-based polymer is crosslinked. In some embodiments, the cyclic oligosaccharide-based polymer is an alkylated derivative. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) cyclic oligosaccharide-based polymer. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) cyclodextrin or an alkylated derivative thereof. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) gamma cyclodextrin or an alkylated derivative thereof.

In some embodiments, the cyclic oligosaccharide-based polymer comprises gamma cyclodextrin. In some embodiments, the gamma cyclodextrin comprises non-crosslinked gamma cyclodextrin. In some embodiments, the gamma cyclodextrin comprises crosslinked gamma cyclodextrin. In some embodiments, the cyclic oligosaccharide-based polymer comprises hydroxypropyl gamma cyclodextrin.

In some embodiments, the alpha cyclodextrin has the following chemical formula:

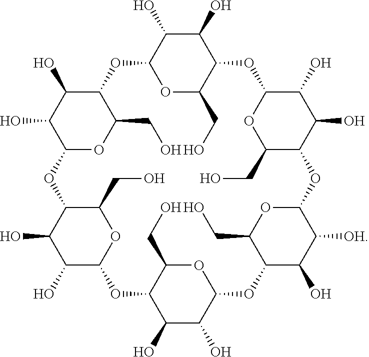 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]1[C@H](O)[C@H]2O)O[C@@H]7CO)O[C@@H]6CO)O[C@@H]5CO)O[C@@H]4CO)O[C@@H]3CO]

In some embodiments, the beta cyclodextrin has the following chemical formula:

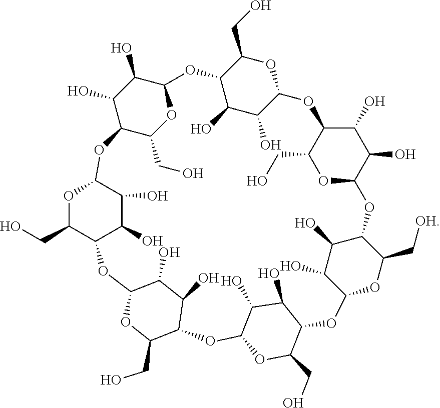 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In some embodiments, the gamma cyclodextrin has the following chemical formula:

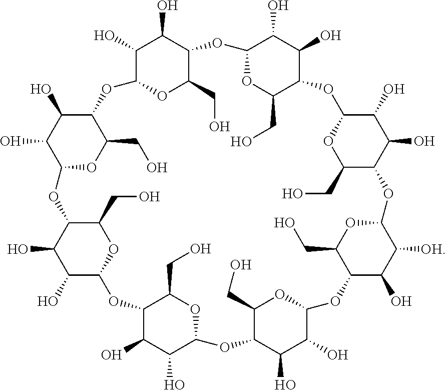 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@H]1[C@H](O)[C@H]2O)O[C@@H]9CO)O[C@@H]8CO)O[C@@H]7CO)O[C@@H]6CO)O[C@@H]5CO)O[C@@H]4CO)O[C@@H]3CO]

In some embodiments, the hydroxypropyl gamma cyclodextrin has the following chemical formula:

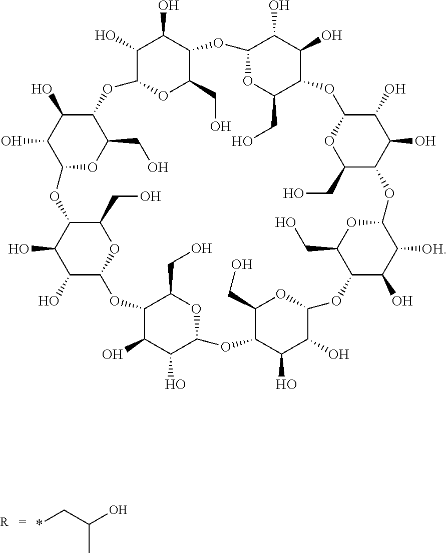

In some embodiments, the ratio of the carnosine/carnitine salts to cyclodextrin is between 1:1 and 10:1 by weight. In some embodiments, the ratio of the carnosine/carnitine salts to cyclodextrin is between 1:1 and 3:1 by weight. In some embodiments, the composition comprises a ratio of salts to cyclodextrin of 1:1 to 1:5 without hyaluronic acid/sodium hyaluronate. In some embodiments, the composition comprises a ratio of salts to alpha- or gamma-cyclodextrin of 1:5 to 5:1 without hyaluronic acid/sodium hyaluronate. In some embodiments, the composition comprises up to 13% (wt) alpha-cyclodextrin without hyaluronic acid/sodium hyaluronate. In some embodiments, the composition comprises up to 23% by weight of gamma-cyclodextrin without hyaluronic acid/sodium hyaluronate. In some embodiments, the composition comprises up to 23% by weight of hydroxyproylgamma cyclodextrin without hyaluronic acid/sodium hyaluronate. In some embodiments, the composition comprises a ratio of salts to beta cyclodextrin of 5:1 to 1:1 without hyaluronic acid/sodium hyaluronate. In some embodiments, the composition comprises up to 1% beta-cyclodextrin without hyaluronic acid/sodium hyaluronate. In some embodiments, the composition comprises 5% salts and 1% beta cyclodextrin without hyaluronic acid/sodium hyaluronate. In some embodiments, the composition comprises up to 10% lactic acid salts without hyaluronic acid/sodium hyaluronate. In some embodiments, the composition comprises up to 10% glycolic acid salts without hyaluronic acid/sodium hyaluronate. In some embodiments, the composition comprises up to 3% salycilic acid salts without hyaluronic acid/sodium hyaluronate.

In some embodiments, the composition comprises a ratio of salts to cyclodextrin to hyaluronic acid/sodium hyaluronate of 10:1:1 to 1:10:10. In some embodiments, the composition comprises a ratio of salts to cyclodextrin to hyaluronic acid/sodium hyaluronate of 5:1:1 to 1:10:10. In some embodiments, the composition comprises a ratio of salts to gammacyclodextrin to hyaluronic acid/sodium hyaluronate of up to 5:1:1. In some embodiments, the composition comprises a ration of salts to cyclodextrin to hyaluronic acid/sodium hyaluronate of up to 16:3:1. In some embodiments, the composition comprises a ratio of salts to cyclodextrin to hyaluronic acid/sodium hyaluronate of up to 7:1:1. In some embodiments, the composition comprises a ratio of salts to cyclodextrin to hyaluronic acid/sodium hyaluronate of up to 7:1:1.5. In some embodiments, the composition comprises up to 1% cyclodextrin and 1% hyaluronic acid/sodium hyaluronate. In some embodiments, the composition comprises up to 1% gammacyclodextrin and 1% hyaluronic acid/sodium hyaluronate. In some embodiments, the composition comprises up to 1% hydroxypropylgamma cyclodextrin and 1% hyaluronic acid/sodium hyaluronate. In some embodiments, the composition comprises up to 0.5% beta-cyclodextrin and 1% hyaluronic acid/sodium hyaluronate. In some embodiments, the composition comprises up to 10% lactic acid salts with hyaluronic acid/sodium hyaluronate. In some embodiments, the composition comprises up to 10% glycolic acid salts with hyaluronic acid/sodium hyaluronate. In some embodiments, the composition comprises up to 3% salycilic acid salts with hyaluronic acid/sodium hyaluronate.

In some embodiments, when salts under 2% concentration are present in the composition, the composition may comprise 0.5% to 1.1% alpha cyclodextrin or hydroxypropylbeta cyclodextrin. In some such embodiments, the composition comprises up to 1% hyaluronic acid/sodium hyaluronate. In some embodiments, when the composition comprises salt concentrations over 3%, the composition may comprise 0.1-1% gamma- or hydroxypropylgamma cyclodextrin. I some such embodiments, the composition comprises up to 1% (such as between 0.1-0.7%) hyaluronic acid/sodium hyaluronate.

In some embodiments, the composition comprises up to 0.8% phenoxyethanol. In some embodiments, the composition comprises 0.1% to 0.8% phenoxyethanol. In some embodiments, the composition comprises up to 0.8% of an aromatic alcohol. In some embodiments, the composition comprises up to 0.8% benzyl alcohol. In some embodiments, the composition comprises 0.1% to 0.8% benzyl alcohol. In some embodiments, the composition comprises up to 0.1% ethylhexylglycerin. In some embodiments, the composition comprises 0.001% to 0.1% ethylhexylglycerin. In some embodiments, the composition comprises up to 0.3% parabens. In some embodiments, the composition comprises up to 0.3% methyl- or propyl-parabens.

In some embodiments, the composition comprises up to 8% carnosine. In some embodiments, the composition comprises up to 8% carnitine. In some embodiments, the composition comprises up to 10% salycilic acid. In some embodiments, the composition comprises up to 10% lactic acid. In some embodiments, the composition comprises up to 10% glycolic acid. In some embodiments, the composition comprises up to 1% cyclodextrin. In some embodiments, the composition comprises up to 5% allantoin. In some embodiments, the composition comprises up to 1% hyaluronic acid. In some embodiments, the composition comprises up to 1% sodium hyaluronate. In some embodiments, the composition comprises up to 10% trimethylglycine.

In some embodiments, the composition has a pH between 5.5 and 7.3 (e.g., pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3 or any value therebetween). In some embodiments, the composition has a pH between band 7 (e.g., pH 6, 6.1, 6.2, 6.3, 6.4, 6.5, 6.6, 6.7, 6.8, 6.9, 7, or any value therebetween).

In some embodiments, the composition for treating eczema comprises by weight of the total composition: 0.5-6% carnosine; 0.3-5% lactic acid; 0.1-1% hydroxypropyl gamma cyclodextrin; 0.5-3% carnitine; 3-10% trimethylglycine; and 0.1-0.8% sodium hyaluronate. In some embodiments, the composition comprises one or more of 0.4-3.5% salicylic acid, 0.2-3% glycolic acid, and 0.5% allantoin.

In some embodiments, the composition for treating eczema comprises 4% carnosine; 1.8% salicylic acid; 0.8% lactic acid; 0.5% glycolic acid; 0.5% hydroxypropyl gamma cyclodextrin; 1% carnitine; 3% trimethylglycine; and 0.2% sodium hyaluronate.

In some embodiments, the composition for treating eczema comprises 2% carnosine; 1.4% lactic acid; 0.2% hydroxypropyl gamma cyclodextrin; 1% carnitine; 7% trimethylglycine; 0.2% sodium hyaluronate; and 0.5% allantoin.

In some embodiments, the composition for treating eczema comprises 5% carnosine; 2.8% lactic acid; 0.3% glycolic acid; 0.5% hydroxypropyl gamma cyclodextrin; 2% carnitine; 7% trimethylglycine; 0.6% sodium hyaluronate; and 0.5% allantoin.

In some embodiments, the composition for treating eczema comprises 4% carnosine; 1.8% salicylic acid; 0.8% lactic acid; 0.5% glycolic acid; 0.5% hydroxypropyl gamma cyclodextrin; 1% carnitine; 3% trimethylglycine; 0.2% sodium hyaluronate; 0.5% glycerin; 8% 1,3-propanediol; 2% pentylene glycol; 0.6% phenoxyethanol; and 0.05% ethylhexylglycerine.

In some embodiments, the composition for treating eczema comprises 2% carnosine; 1.4% lactic acid; 0.2% hydroxypropyl gamma cyclodextrin; 1% carnitine; 7% trimethylglycine; 0.2% sodium hyaluronate; 0.5% allantoin; 0.5% glycerin; 8% 1,3-propanediol; 2% pentylene glycol; 0.6% phenoxyethanol; 0.05% ethylhexylglycerine; and 0.5% allantoin.

In some embodiments, the composition for treating eczema comprises 5% carnosine; 2.8% lactic acid; 0.3% glycolic acid; 0.5% hydroxypropyl gamma cyclodextrin; 2% carnitine; 7% trimethylglycine; 0.6% sodium hyaluronate; 0.5% glycerin; 8% 1,3-propanediol; 2% pentylene glycol; 0.6% phenoxyethanol; 0.05% ethylhexylglycerine; and 0.5% allantoin.

In some embodiments, the compositions for treating eczema further comprises at least one pharmaceutically acceptable carrier or excipient.

In some embodiments, the compositions for treating eczema further comprises at least one of: a pH buffer; a humectant; a nonionic surfactant; and a preservative.

In some embodiments, the composition for treating eczema comprises one or more polymers. In some embodiments, the composition for treating eczema comprises one or more of hyaluronic acid (of either low, medium, or high molecular weights), polyacrylate crosspolymer-6, xanthan gum, sodium carbomer, hydroxyethyl acrylate, sodium acryloyldimethyl taurate copolymer, and polyacrylate-13.

In some embodiments, the composition for treating eczema is a patch, a cream, an ointment, a powder, an aerosol spray, or a hydrogel. In some embodiments, the composition for treating eczema is impregnated into a dressing.

### Dandruff Formulations

According to some aspects, the present disclosure provides a composition for treatment of dandruff, comprising one or more of carnosine; salicylic acid; lactic acid; glycolic acid, cyclodextrin; carnitine; trimethylglycine; and sodium hyaluronate. In some embodiments, the carnosine and/or carnitine form a salt via reaction with an acid. In some embodiments, the composition further comprises one or more of salicylic acid and glycolic acid.

In some embodiments, the carnosine comprises L-carnosine, D-carnosine, acetyl-carnosine, a derivative thereof, or a combination thereof. In some embodiments, the antioxidant comprises L-carnosine. In some embodiments, the carnitine comprises L-carnitine, D-carnitine, acetyl-carnitine, propionyl-L-carnitine, a derivative thereof, or a combination thereof. In some embodiments, the compositions for treating dandruff comprises, by weight of the total composition, 0.1%-10% (0.2, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnosine. In some embodiments, the compositions for treating dandruff comprises, by weight of the total composition, 0.5%-10% (0.1%-10% (0.2, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnitine.

In some embodiments, the composition for treating dandruff comprises one or more of carnosine, carnitine, and cyclodextrin. In some embodiments, the composition for treating dandruff comprises comprising L-carnosine and gamma cyclodextrin.

In some embodiments, the composition comprises a cyclic oligosaccharide-based polymer, such as an alpha cyclodextrin, beta cyclodextrin, a gamma cyclodextrin, hydroxypropyl gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glocosyl-β-cyclodextrin, maltosyl-β-cyclodextrin or any derivative thereof, and any combination thereof. In some embodiments, the cyclic oligosaccharide-based polymer is non-crosslinked. In some embodiments, the cyclic oligosaccharide-based polymer is crosslinked. In some embodiments, the cyclic oligosaccharide-based polymer is an alkylated derivative. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) cyclic oligosaccharide-based polymer. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) cyclodextrin or an alkylated derivative thereof. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) gamma cyclodextrin or an alkylated derivative thereof.

In some embodiments, the cyclic oligosaccharide-based polymer comprises gamma cyclodextrin. In some embodiments, the gamma cyclodextrin comprises non-crosslinked gamma cyclodextrin. In some embodiments, the gamma cyclodextrin comprises crosslinked gamma cyclodextrin. In some embodiments, the cyclic oligosaccharide-based polymer comprises hydroxypropyl gamma cyclodextrin.

In some embodiments, the alpha cyclodextrin has the following chemical formula:

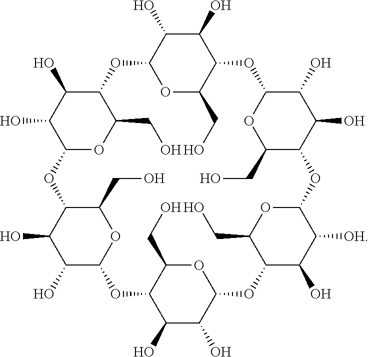 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]1[C@H](O)[C@H]2O)O[C@@H]7CO)O[C@@H]6CO)O[C@@H]5CO)O[C@@H]4CO)O[C@@H]3CO]

In some embodiments, the beta cyclodextrin has the following chemical formula:

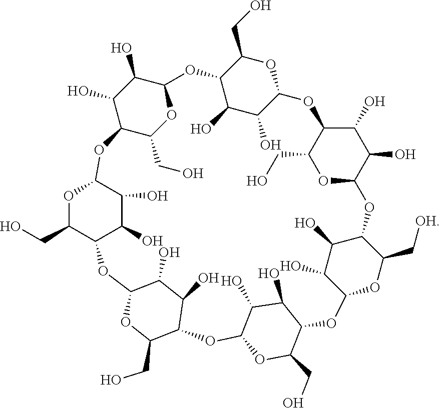 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In some embodiments, the gamma cyclodextrin has the following chemical formula:

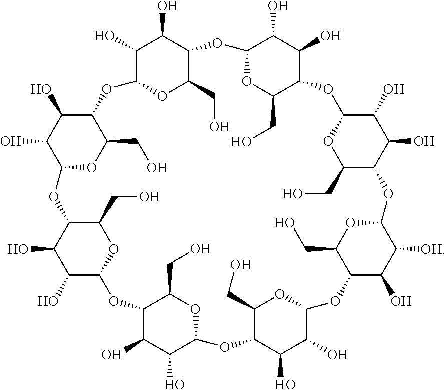 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@H]1[C@H](O)[C@H]2O)O[C@@H]9CO)O[C@@H]8CO)O[C@@H]7CO)O[C@@H]6CO)O[C@@H]5CO)O[C@@H]4CO)O[C@@H]3CO]

In some embodiments, the hydroxypropyl gamma cyclodextrin has the following chemical formula:

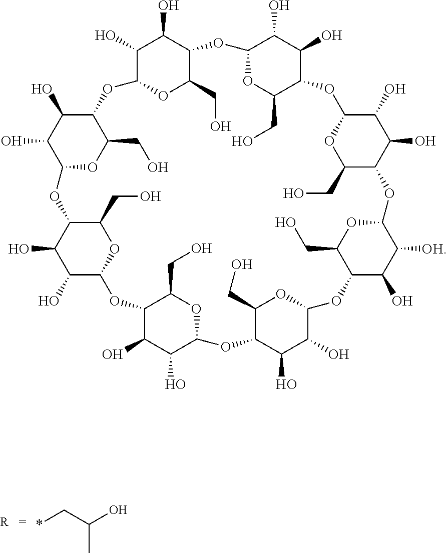

In some embodiments, the ratio of the carnosine/carnitine salts to cyclodextrin is between 1:1 and 10:1 by weight. In some embodiments, the ratio of the carnosine/carnitine salts to cyclodextrin is between 1:1 and 3:1 by weight. In some embodiments, the composition comprises a ratio of salts to cyclodextrin of 1:1 to 1:5 without hyaluronic acid/sodium hyaluronate. In some embodiments, the composition comprises a ratio of salts to alpha- or gamma-cyclodextrin of 1:5 to 5:1 without hyaluronic acid/sodium hyaluronate. In some embodiments, the composition comprises up to 13% (wt) alpha-cyclodextrin without hyaluronic acid/sodium hyaluronate. In some embodiments, the composition comprises up to 23% by weight of gamma-cyclodextrin without hyaluronic acid/sodium hyaluronate. In some embodiments, the composition comprises up to 23% by weight of hydroxyproylgamma cyclodextrin without hyaluronic acid/sodium hyaluronate. In some embodiments, the composition comprises a ratio of salts to beta cyclodextrin of 5:1 to 1:1 without hyaluronic acid/sodium hyaluronate. In some embodiments, the composition comprises up to 1% beta-cyclodextrin without hyaluronic acid/sodium hyaluronate. In some embodiments, the composition comprises 5% salts and 1% beta cyclodextrin without hyaluronic acid/sodium hyaluronate. In some embodiments, the composition comprises up to 10% lactic acid salts without hyaluronic acid/sodium hyaluronate. In some embodiments, the composition comprises up to 10% glycolic acid salts without hyaluronic acid/sodium hyaluronate. In some embodiments, the composition comprises up to 3% salycilic acid salts without hyaluronic acid/sodium hyaluronate.

In some embodiments, the composition comprises a ratio of salts to cyclodextrin to hyaluronic acid/sodium hyaluronate of 10:1:1 to 1:10:10. In some embodiments, the composition comprises a ratio of salts to cyclodextrin to hyaluronic acid/sodium hyaluronate of 5:1:1 to 1:10:10. In some embodiments, the composition comprises a ratio of salts to gammacyclodextrin to hyaluronic acid/sodium hyaluronate of up to 5:1:1. In some embodiments, the composition comprises a ration of salts to cyclodextrin to hyaluronic acid/sodium hyaluronate of up to 16:3:1. In some embodiments, the composition comprises a ratio of salts to cyclodextrin to hyaluronic acid/sodium hyaluronate of up to 7:1:1. In some embodiments, the composition comprises a ratio of salts to cyclodextrin to hyaluronic acid/sodium hyaluronate of up to 7:1:1.5. In some embodiments, the composition comprises up to 1% cyclodextrin and 1% hyaluronic acid/sodium hyaluronate. In some embodiments, the composition comprises up to 1% gammacyclodextrin and 1% hyaluronic acid/sodium hyaluronate. In some embodiments, the composition comprises up to 1% hydroxypropylgamma cyclodextrin and 1% hyaluronic acid/sodium hyaluronate. In some embodiments, the composition comprises up to 0.5% beta-cyclodextrin and 1% hyaluronic acid/sodium hyaluronate. In some embodiments, the composition comprises up to 10% lactic acid salts with hyaluronic acid/sodium hyaluronate. In some embodiments, the composition comprises up to 10% glycolic acid salts with hyaluronic acid/sodium hyaluronate. In some embodiments, the composition comprises up to 3% salycilic acid salts with hyaluronic acid/sodium hyaluronate.

In some embodiments, when salts under 2% concentration are present in the composition, the composition may comprise 0.5% to 1.1% alpha cyclodextrin or hydroxypropylbeta cyclodextrin. In some such embodiments, the composition comprises up to 1% hyaluronic acid/sodium hyaluronate. In some embodiments, when the composition comprises salt concentrations over 3%, the composition may comprise 0.1-1% gamma- or hydroxypropylgamma cyclodextrin. I some such embodiments, the composition comprises up to 1% (such as between 0.1-0.7%) hyaluronic acid/sodium hyaluronate.

In some embodiments, the composition comprises up to 0.8% phenoxyethanol. In some embodiments, the composition comprises 0.1% to 0.8% phenoxyethanol. In some embodiments, the composition comprises up to 0.8% of an aromatic alcohol. In some embodiments, the composition comprises up to 0.8% benzyl alcohol. In some embodiments, the composition comprises 0.1% to 0.8% benzyl alcohol. In some embodiments, the composition comprises up to 0.1% ethylhexylglycerin. In some embodiments, the composition comprises 0.001% to 0.1% ethylhexylglycerin. In some embodiments, the composition comprises up to 0.3% parabens. In some embodiments, the composition comprises up to 0.3% methyl- or propyl-parabens.

In some embodiments, the composition comprises up to 8% carnosine. In some embodiments, the composition comprises up to 8% carnitine. In some embodiments, the composition comprises up to 10% salycilic acid. In some embodiments, the composition comprises up to 10% lactic acid. In some embodiments, the composition comprises up to 10% glycolic acid. In some embodiments, the composition comprises up to 1% cyclodextrin. In some embodiments, the composition comprises up to 5% allantoin. In some embodiments, the composition comprises up to 1% hyaluronic acid. In some embodiments, the composition comprises up to 1% sodium hyaluronate. In some embodiments, the composition comprises up to 10% trimethylglycine.

In some embodiments, the composition has a pH between 5.5 and 7.3 (e.g., pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3 or any value therebetween). In some embodiments, the composition has a pH between band 7 (e.g., pH 6, 6.1, 6.2, 6.3, 6.4, 6.5, 6.6, 6.7, 6.8, 6.9, 7, or any value therebetween).

In some embodiments, the composition for treating dandruff comprises by weight of the total composition one or more of 0.5-6% carnosine; 0.4-3.5% salicylic acid; 0.3-5% lactic acid; 0.2-3% glycolic acid; 0.1-1% cyclodextrin; 0.5-3% carnitine; 3-10% trimethylglycine; and 0.1-0.8% sodium hyaluronate. In some embodiments, the composition comprises one or more of 0.4-3.5% salicylic acid, 0.2-3% glycolic acid, and 0.5% allantoin.

In some embodiments, the composition for treating dandruff comprises one or more of 0.5-6% carnosine; 0.4-3.5% salicylic acid; 0.3-5% lactic acid; 0.2-3% glycolic acid, 0.1-1% cyclodextrin; 0.5-3% carnitine; 3-10% trimethylglycine; and 0.1-0.8% sodium hyaluronate.

In some embodiments, the composition for treating dandruff comprises one or more of 4% carnosine; 1.8% salicylic acid; 0.8% lactic acid; 0.5% glycolic acid, 0.5% cyclodextrin; 1% carnitine; 0.5% glycerin; 8% 1,3-propanediol; 2% pentylene glycol; 3% trimethylglycine; 0.2% sodium hyaluronate; 0.6% phenoxyethanol; 0.05% ethylhexylglycerin.

In some embodiments, the compositions for treating dandruff further comprises at least one pharmaceutically acceptable carrier or excipient.

In some embodiments, the compositions for treating dandruff further comprises at least one of: a pH buffer; a humectant; a nonionic surfactant; and a preservative.

In some embodiments, the composition for treating dandruff comprises one or more polymers. In some embodiments, the composition for treating dandruff comprises one or more of hyaluronic acid (of either low, medium, or high molecular weights), polyacrylate crosspolymer-6, xanthan gum, sodium carbomer, hydroxyethyl acrylate, sodium acryloyldimethyl taurate copolymer, and polyacrylate-13.

In some embodiments, the composition for treating dandruff is a cream, an ointment, a powder, an aerosol spray, or a hydrogel.

### Acne Formulations

According to some aspects, the present disclosure provides a composition for treatment of acne comprising one or more of carnosine; salicylic acid; lactic acid; glycolic acid, cyclodextrin; carnitine; trimethylglycine; and sodium hyaluronate. In some embodiments, the carnosine and/or carnitine form a salt via reaction with an acid. In some embodiments, the composition further comprises one or more of salicylic acid, lactic acid, and glycolic acid.

In some embodiments, the carnosine comprises L-carnosine, D-carnosine, acetyl-carnosine, a derivative thereof, or a combination thereof. In some embodiments, the antioxidant comprises L-carnosine. In some embodiments, the carnitine comprises L-carnitine, D-carnitine, acetyl-carnitine, propionyl-L-carnitine, a derivative thereof, or a combination thereof. In some embodiments, the compositions for treating acne comprises, by weight of the total composition, 0.1%-10% (0.2, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnosine. In some embodiments, the compositions for treating acne comprises, by weight of the total composition, 0.5%-10% (0.1%-10% (0.2, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) carnitine.

In some embodiments, the composition for treating acne comprises one or more of carnosine, carnitine, and cyclodextrin. In some embodiments, the composition for treating acne comprises comprising L-carnosine and gamma cyclodextrin.

In some embodiments, the composition comprises a cyclic oligosaccharide-based polymer, such as an alpha cyclodextrin, beta cyclodextrin, a gamma cyclodextrin, hydroxypropyl gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glocosyl-β-cyclodextrin, maltosyl-β-cyclodextrin or any derivative thereof, and any combination thereof. In some embodiments, the cyclic oligosaccharide-based polymer is non-crosslinked. In some embodiments, the cyclic oligosaccharide-based polymer is crosslinked. In some embodiments, the cyclic oligosaccharide-based polymer is an alkylated derivative. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) cyclic oligosaccharide-based polymer. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) cyclodextrin or an alkylated derivative thereof. In some embodiments, the compositions for treating an inflammatory skin condition comprises, by weight of the total composition, 0.01%-10% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10% or any value therebetween) gamma cyclodextrin or an alkylated derivative thereof.

In some embodiments, the cyclic oligosaccharide-based polymer comprises gamma cyclodextrin. In some embodiments, the gamma cyclodextrin comprises non-crosslinked gamma cyclodextrin. In some embodiments, the gamma cyclodextrin comprises crosslinked gamma cyclodextrin. In some embodiments, the cyclic oligosaccharide-based polymer comprises hydroxypropyl gamma cyclodextrin.

In some embodiments, the alpha cyclodextrin has the following chemical formula:

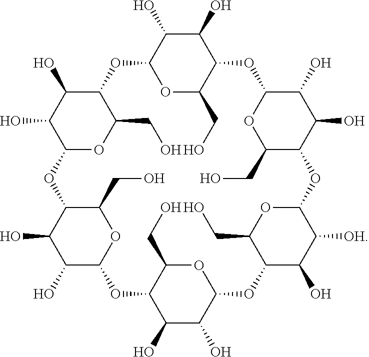 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]1[C@H](O)[C@H]2O)O[C@@H]7CO)O[C@@H]6CO)O[C@@H]5CO)O[C@@H]4CO)O[C@@H]3CO]

In some embodiments, the beta cyclodextrin has the following chemical formula:

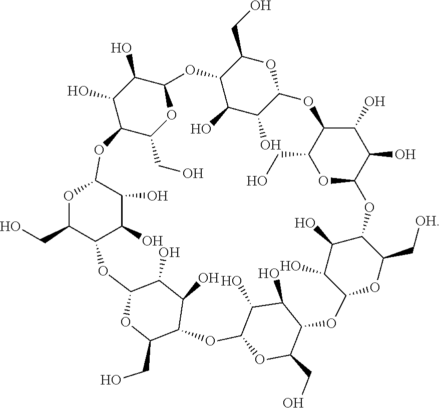 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In some embodiments, the gamma cyclodextrin has the following chemical formula:

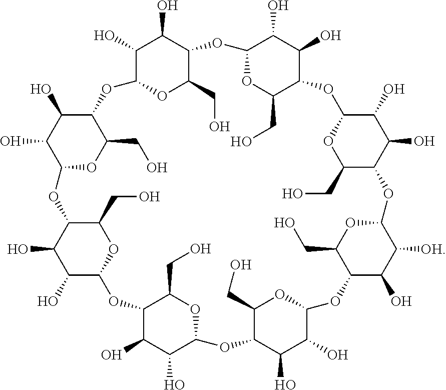 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@H]1[C@H](O)[C@H]2O)O[C@@H]9CO)O[C@@H]8CO)O[C@@H]7CO)O[C@@H]6CO)O[C@@H]5CO)O[C@@H]4CO)O[C@@H]3CO]

In some embodiments, the hydroxypropyl gamma cyclodextrin has the following chemical formula:

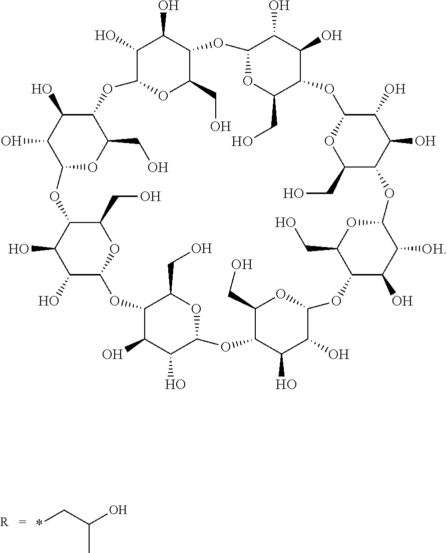

In some embodiments, the ratio of the carnosine/carnitine salts to cyclodextrin is between 1:1 and 10:1 by weight. In some embodiments, the ratio of the carnosine/carnitine salts to cyclodextrin is between 1:1 and 3:1 by weight. In some embodiments, the composition comprises a ratio of salts to cyclodextrin of 1:1 to 1:5 without hyaluronic acid/sodium hyaluronate. In some embodiments, the composition comprises a ratio of salts to alpha- or gamma-cyclodextrin of 1:5 to 5:1 without hyaluronic acid/sodium hyaluronate. In some embodiments, the composition comprises up to 13% (wt) alpha-cyclodextrin without hyaluronic acid/sodium hyaluronate. In some embodiments, the composition comprises up to 23% by weight of gamma-cyclodextrin without hyaluronic acid/sodium hyaluronate. In some embodiments, the composition comprises up to 23% by weight of hydroxyproylgamma cyclodextrin without hyaluronic acid/sodium hyaluronate. In some embodiments, the composition comprises a ratio of salts to beta cyclodextrin of 5:1 to 1:1 without hyaluronic acid/sodium hyaluronate. In some embodiments, the composition comprises up to 1% beta-cyclodextrin without hyaluronic acid/sodium hyaluronate. In some embodiments, the composition comprises 5% salts and 1% beta cyclodextrin without hyaluronic acid/sodium hyaluronate. In some embodiments, the composition comprises up to 10% lactic acid salts without hyaluronic acid/sodium hyaluronate. In some embodiments, the composition comprises up to 10% glycolic acid salts without hyaluronic acid/sodium hyaluronate. In some embodiments, the composition comprises up to 3% salycilic acid salts without hyaluronic acid/sodium hyaluronate.

In some embodiments, the composition comprises a ratio of salts to cyclodextrin to hyaluronic acid/sodium hyaluronate of 10:1:1 to 1:10:10. In some embodiments, the composition comprises a ratio of salts to cyclodextrin to hyaluronic acid/sodium hyaluronate of 5:1:1 to 1:10:10. In some embodiments, the composition comprises a ratio of salts to gammacyclodextrin to hyaluronic acid/sodium hyaluronate of up to 5:1:1. In some embodiments, the composition comprises a ration of salts to cyclodextrin to hyaluronic acid/sodium hyaluronate of up to 16:3:1. In some embodiments, the composition comprises a ratio of salts to cyclodextrin to hyaluronic acid/sodium hyaluronate of up to 7:1:1. In some embodiments, the composition comprises a ratio of salts to cyclodextrin to hyaluronic acid/sodium hyaluronate of up to 7:1:1.5. In some embodiments, the composition comprises up to 1% cyclodextrin and 1% hyaluronic acid/sodium hyaluronate. In some embodiments, the composition comprises up to 1% gammacyclodextrin and 1% hyaluronic acid/sodium hyaluronate. In some embodiments, the composition comprises up to 1% hydroxypropylgamma cyclodextrin and 1% hyaluronic acid/sodium hyaluronate. In some embodiments, the composition comprises up to 0.5% beta-cyclodextrin and 1% hyaluronic acid/sodium hyaluronate. In some embodiments, the composition comprises up to 10% lactic acid salts with hyaluronic acid/sodium hyaluronate. In some embodiments, the composition comprises up to 10% glycolic acid salts with hyaluronic acid/sodium hyaluronate. In some embodiments, the composition comprises up to 3% salycilic acid salts with hyaluronic acid/sodium hyaluronate.

In some embodiments, when salts under 2% concentration are present in the composition, the composition may comprise 0.5% to 1.1% alpha cyclodextrin or hydroxypropylbeta cyclodextrin. In some such embodiments, the composition comprises up to 1% hyaluronic acid/sodium hyaluronate. In some embodiments, when the composition comprises salt concentrations over 3%, the composition may comprise 0.1-1% gamma- or hydroxypropylgamma cyclodextrin. I some such embodiments, the composition comprises up to 1% (such as between 0.1-0.7%) hyaluronic acid/sodium hyaluronate.

In some embodiments, the composition comprises up to 0.8% phenoxyethanol. In some embodiments, the composition comprises 0.1% to 0.8% phenoxyethanol. In some embodiments, the composition comprises up to 0.8% of an aromatic alcohol. In some embodiments, the composition comprises up to 0.8% benzyl alcohol. In some embodiments, the composition comprises 0.1% to 0.8% benzyl alcohol. In some embodiments, the composition comprises up to 0.1% ethylhexylglycerin. In some embodiments, the composition comprises 0.001% to 0.1% ethylhexylglycerin. In some embodiments, the composition comprises up to 0.3% parabens. In some embodiments, the composition comprises up to 0.3% methyl- or propyl-parabens.

In some embodiments, the composition comprises up to 8% carnosine. In some embodiments, the composition comprises up to 8% carnitine. In some embodiments, the composition comprises up to 10% salycilic acid. In some embodiments, the composition comprises up to 10% lactic acid. In some embodiments, the composition comprises up to 10% glycolic acid. In some embodiments, the composition comprises up to 1% cyclodextrin. In some embodiments, the composition comprises up to 5% allantoin. In some embodiments, the composition comprises up to 1% hyaluronic acid. In some embodiments, the composition comprises up to 1% sodium hyaluronate. In some embodiments, the composition comprises up to 10% trimethylglycine.

In some embodiments, the composition has a pH between 5.5 and 7.3 (e.g., pH 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3 or any value therebetween). In some embodiments, the composition has a pH between band 7 (e.g., pH 6, 6.1, 6.2, 6.3, 6.4, 6.5, 6.6, 6.7, 6.8, 6.9, 7, or any value therebetween).

In some embodiments, the composition for treating acne comprises by weight of the total composition one or more of 0.5-6% carnosine; 0.4-3.5% salicylic acid; 0.3-5% lactic acid; 0.2-3% glycolic acid; 0.1-1% cyclodextrin; 0.5-3% carnitine; 3-10% trimethylglycine; and 0.1-0.8% sodium hyaluronate. In some embodiments, the composition comprises one or more of 0.4-3.5% salicylic acid, 0.2-3% glycolic acid, and 0.5% allantoin.

In some embodiments, the composition for treating acne comprises one or more of 0.5-6% carnosine; 0.4-3.5% salicylic acid; 0.3-5% lactic acid; 0.2-3% glycolic acid, 0.1-1% cyclodextrin; 0.5-3% carnitine; 3-10% trimethylglycine; and 0.1-0.8% sodium hyaluronate.

In some embodiments, the composition for treating acne comprises one or more of 4% carnosine; 1.8% salicylic acid; 0.8% lactic acid; 0.5% glycolic acid, 0.5% cyclodextrin; 1% carnitine; 0.5% glycerin; 8% 1,3-propanediol; 2% pentylene glycol; 3% trimethylglycine; 0.2% sodium hyaluronate; 0.6% phenoxyethanol; 0.05% ethylhexylglycerin.

In some embodiments, the composition for acne treatment comprises one or more of 4% carnosine; 1.8% salicylic acid; 0.8% lactic acid; 1% carnitine; 0.5% glycolic acid; 0.5% cyclodextrin; 0.5% glycerin; 8% 1,3-propanediol; 2% pentylene glycol; 3% trimethylglycine; 0.2% sodium hyaluronate; 0.6% phenoxyethanol; 0.05% ethylhexylglycerin.

In some embodiments, the compositions for treating acne further comprises at least one pharmaceutically acceptable carrier or excipient.

In some embodiments, the compositions for treating acne further comprises at least one of: a pH buffer; a humectant; a nonionic surfactant; and a preservative.

In some embodiments, the composition for treating acne comprises one or more polymers. In some embodiments, the composition for treating acne comprises one or more of hyaluronic acid (of either low, medium, or high molecular weights), polyacrylate crosspolymer-6, xanthan gum, sodium carbomer, hydroxyethyl acrylate, sodium acryloyldimethyl taurate copolymer, and polyacrylate-13.

In some embodiments, the composition for treating acne is a patch, a cream, an ointment, a powder, an aerosol spray, or a hydrogel. In some embodiments, the composition for treating acne is impregnated into a dressing.

### Inflammatory Skin Condition Treatment Composition Ingredients

In some embodiments, the compositions for treating an inflammatory skin condition (such as eczema, dandruff, and acne) comprises 0.05%-3% (e.g., 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3%, or any value therebetween) pH buffer. In some embodiments, the compositions for treating an inflammatory skin condition comprises 3%-30% (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30%, or any value therebetween) humectant. In some embodiments, the compositions for treating an inflammatory skin condition comprises 5%-15% (5, 5.5, 6, 6.5, 7, 7.5, 8, 8.5, 9, 9.5, 10, 10.5, 11, 11.5, 12, 12.5, 13, 13.5, 14, 14.5, 15%, or any value therebetween) nonionic surfactant. In some embodiments, the compositions for treating an inflammatory skin condition comprises 0.05%-1.5% (e.g., 0.05, 0.1, 0.2, 0.3, 0.4, 0.5, 0.6, 0.7, 0.8, 0.9, 1, 1.1, 1.2, 1.3, 1.4, 1.5, or any value therebetween) preservative.

In some embodiments, the pH buffer comprises at least one of citric acid, magnesium citrate, magnesium sulfate, sodium citrate or sodium sulfate. In some embodiments, the pH buffer comprises citric acid.

In some embodiments, the humectant comprises at least one of an amino acid, aloe vera extract, a fatty acid, hyaluronic acid (HA), collagen, silicone, a disaccharide (e.g., sucrose or trehalose), maltitol, erythrol, sorbitol, glycerin, propanediol, propylene glycol, glycerin or any other glycol/diol. In some embodiments, the humectant comprises glycerin and/or HA. In some embodiments, HA is swapped for any other heavy molecular weight polymer such as collagen or a derivative thereof. In some embodiments, collagen is interchangeable with HA or can be combined with HA, e.g., 0.6% HA combined with 0.5% collagen. In some embodiments, the composition comprises 0.1-5% of HA, 0.1-5% of collagen or derivatives thereof, or 0.1-5% of HA and collagen or a derivative thereof combined. In some embodiments, the compositions for treating an inflammatory skin condition comprises 3-30% (e.g., 3, 4, 5, 6, 7, 8, 9, 10, 11, 12, 13, 14, 15, 16, 17, 18, 19, 20, 21, 22, 23, 24, 25, 26, 27, 28, 29, 30% or any number therebetween) of propanediol, propylene glycol, glycerin or any other glycol/diol, or a combination thereof.

In some embodiments, the nonionic surfactant comprises at least one of tearyl alcohol, cetearyl alcohol, or a combination of both. In some embodiments, the nonionic surfactant comprises tearyl alcohol. In some embodiments, the nonionic surfactant comprises cetearyl alcohol. In some embodiments, the compositions for treating an inflammatory skin condition comprises 3-30% of a nonionic surfactant.

In some embodiments, the preservative comprises one or more of parabens, alkyl parabens, aromatic alcohols, glycols, and chelating agents (EDTA, sodium phytate). In some embodiments, the preservative comprises at least one of sodium benzoate, benzyl alcohol, sorbic acid, triclosan, phenoxyisopropanol, diazolidinyl urea, bronopol, Alkyl (C12-22) trimethyl ammonium bromide, Alkyl (C12-22) trimethyl ammonium chloride, Benzalkonium chloride, Benzalkonium bromide, Benzalkonium saccharinate, ethylhexylglycerin, or phenoxyethanol. In some embodiments, the preservative comprises phenoxyethanol or ethylhexylglycerin or both, optionally at 0.1%-1.5% of the composition. In some embodiments, the phenoxyethanol or ethylhexylglycerin can be swapped for parabens or other aromatic alcohols such as benzyl alcohol, optionally at 0.1%-1.5% of the composition.

In some embodiments, the compositions for treating an inflammatory skin condition further comprises at least one of the following constituents: sunflower oil, Tegomuls 90 S, cetyl alcohol, cetyl palmitate, Polysorbate 80, methyl 4-hydroxybenzoate, propyl 4-hydroxybenzoate, sesame oil, wool wax, yellow wax, wool wax alcohols, glycerol, glycerol ester, palmitic acid ester, vaseline, cocoa butter, liquid wax, olive oil or distilled water.

In some embodiments, the compositions for treating an inflammatory skin condition further comprises at least one constituent selected from the group consisting of an antibiotic agent, a free radical generating agent, an antifungal agent, an antiviral agent, a nucleoside-analog reverse transcriptase inhibitor, a non-nucleoside reverse transcriptase inhibitor, a protease inhibitor, an immunosuppressant, an anti-inflammatory agent, a retinoid agent, a tar agent, an antihistamine agent, and an antipruritic agent.

### Formulations

In some embodiments, the composition is lyophilized. In some embodiments, the composition is spray dried to form a sterile powder. In some embodiments, the composition for treating an inflammatory skin condition is a patch, a cream, an ointment, a powder, an aerosol spray, or a hydrogel. In some embodiments, the composition for treating an inflammatory skin condition is impregnated into a dressing. In some embodiments, the composition for treating an inflammatory skin condition is a cream comprising an emulsifier that is not sensitive to salts. In some embodiments, the emulsifiers comprise one or more of Ceteareth 20, Cetearyl Alcohol, Cetyl alcohol, Cetyl esters, PEG 100 Stearate, and Glyceryl Stearate. In some embodiments, the emulsifiers are present in an amount of 0.5-5%.

According to some embodiments, the compositions disclosed herein are in a topical formulation suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter.

Topical formulations include those in which any other active ingredient(s) is (are) dissolved or dispersed in a dermatological vehicle known in the art (e.g., aqueous or nonaqueous gels, ointments, water-in-oil or oil-in-water emulsions). Constituents of such vehicles can comprise water, aqueous buffer solutions, non-aqueous solvents (e.g., ethanol, isopropanol, benzyl alcohol, 2-(2-ethoxyethoxy) ethanol, propylene glycol, propylene glycol monolaurate, glycofurol or glycerol), oils (e.g., a mineral oil such as a liquid paraffin, natural or synthetic triglycerides, or silicone oils such as dimethicone). Depending, inter alia, upon the nature of the formulation as well as its intended use and site of application, the dermatological vehicle employed can contain one or more components (e.g., when the formulation is an aqueous gel, components in addition to water) selected from the following list: a solubilizing agent or solvent (e.g., a β-cyclodextrin, such as hydroxypropyl, or an alcohol or polyol such as ethanol, propylene glycol or glycerol); a thickening agent (e.g., hydroxyethylcellulose, hydroxypropylcellulose, carboxymethylcellulose or carbomer); a gelling agent (e.g., a polyoxyethylene-polyoxypropylene copolymer); a preservative (e.g., benzyl alcohol, benzalkonium chloride, chlorhexidine, chlorbutol, a benzoate, potassium sorbate or EDTA or salt thereof); and pH buffering agent(s) (e.g., a mixture of dihydrogen phosphate and hydrogen phosphate salts, or a mixture of citric acid and a hydrogen phosphate salt).

A pharmaceutically acceptable carrier can also be incorporated in the formulation of the present disclosure and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted, interaction with other components of the formulation. “Carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

According to certain embodiments, the formulations disclosed herein may comprise a film former. A film former, when it dries, forms a protective film over the site of application. The film former inhibits removal of the active ingredient and keeps it in contact with the site being treated. An example of a film former is Flexible Collodion, US P. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which particles, including the active agent, are present in a water or alcohol base. Lotions are usually suspensions of solids, and in some embodiments, comprise a liquid oily emulsion of the oil-in-water type. In some embodiments, lotion formulations are used herein for treating large body areas, because of the ease of applying a more fluid composition. It is generally necessary that the insoluble matter in a lotion be finely-divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize, or preserve the solute. Common examples of solvents used in preparing solutions are ethanol, water, propylene glycol or any other acceptable vehicles. As is well known, gels are semisolid, suspension-type systems. Single-phase gels contain organic macromolecules distributed substantially uniformly throughout the carrier liquid, which is typically aqueous, but also, in some embodiments, contains an alcohol, and, optionally, an oil. In some embodiments, “organic macromolecules,” are used, i.e., gelling agents, are cross-linked acrylic acid polymers such as the “carbomer” family of polymers, e.g., carboxypolyalkylenes that can be obtained commercially under the Carbopol trademark. In other embodiments, hydrophilic polymers are used such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Enhancers are those lipophilic co-enhancers typically referred to as “plasticizing” enhancers, i.e., enhancers that have a molecular weight in the range of about 150 to 1000, an aqueous solubility of less than about 1 wt. %, in some embodiments less than about 0.5 wt. %, and most in some embodiments less than about 0.2 wt. %. The Hildebrand solubility parameter 6 of plasticizing enhancers is in the range of about 2.5 to about 10, in some embodiments in the range of about 5 to about 10. In some embodiments lipophilic enhancers are fatty esters, fatty alcohols, and fatty ethers. Examples of specific fatty acid esters include methyl laurate, ethyl oleate, propylene glycol nionolaurace, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, in some embodiments, a C2-C4 alkane diol or triol, are substituted with one or two fatty ether substituents. Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995)(incorporated herein by reference).

Various other additives can be included in the compositions of the present disclosure in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in the formulations of the disclosure are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), a-tocopherol (Vitamin E), 0-tocopherol, gamma-tocopherol, delta-tocopherol, epsilon-tocopherol, zeta-tocopherol, Z GAMMA-tocopherol, eta-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), a-tocopherol (Vitamin E), beta-tocopherol, gamma-tocopherol, delta-tocopherol, epsilon-tocopherol, zeta-tocopherol, zeta 2-tocopherol, eta-tocopherol, and retinol (Vitamin A), and/or pharmaceutically acceptable salts, esters, amides, or other derivatives thereof. In some embodiments the tocopherol compound is a-tocopherol. Additional agents include those that are capable of improving oxygen supply in skin tissue, as described, for example, in Gross, et al, WO 94/00098 and Gross, et al, WO 94/00109, both assigned to Lancaster Group AG (incorporated herein by reference). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycinnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene. See, Title 21. Chapter 1. Subchapter D. Part 352. “Sunscreen drug products for over-the-counter human use” incorporated herein in its entirety.

Other embodiments can include a variety of non-carcinogenic, non-irritating healing materials that facilitate treatment with the formulations of the disclosure. Such healing materials can include nutrients, minerals, vitamins, electrolytes, enzymes, herbs, plant extracts, glandular or animal extracts, or safe therapeutic agents that can be added to the formulation to facilitate the healing of dermal disorders. The amounts of these various additives are those conventionally used in the cosmetics field, and range, for example, from about 0.010% to about 20% of the total weight of the topical formulation.

The formulations of the disclosure can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds.

The formulations can also contain irritation-mitigating additives to minimize or eliminate the possibility of skin irritation or skin damage resulting from the chemical entity to be administered, or other components of the composition. Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt. %, more typically not more than about 5 wt. %, of the formulation.

Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites. Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycinnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The pharmaceutical compositions of the disclosure comprise one or more active ingredients, e.g. therapeutic agents, in admixture with one or more pharmaceutically-acceptable diluents or carriers and, optionally, one or more other compounds, drugs, ingredients and/or materials. Regardless of the route of administration selected, the agents/compounds of the present disclosure are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the disclosure must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the disclosure may, optionally, contain additional ingredients and/or materials commonly used in pharmaceutical and/or over-the-counter compositions. These ingredients and materials are well known in the art and non-limiting examples include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Dosage forms for the topical or transdermal administration include powders, sprays, ointments, pastes, creams, lotions, gels, solutions, patches, and drops. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

In some embodiments, the compositions disclosed herein may be combined with an active drug substance that is potentially irritating to a subject's skin, such as alpha hydroxy acids, retinoic acids, benzoyl peroxide, calcipotriene, calcineurin inhibitors, sunscreens, sunblocks, bleaching agents, depilatories, antiperspirants, or combinations thereof. In some embodiments, the active drug substance may be anti-rosacea agents such as metronidazole, precipitated sulfur, sodium sulfacetamide, or azelaic acid; antibacterial agents (antibiotics) such as clindamycin phosphate, erythromycin, or antibiotics from the tetracycline family; antimycobacterial agents such as dapsone; other antiacne agents such as retinoids, or benzoyl peroxide; antiparasitic agents such as metronidazole, permethrin, crotamiton, thiabendazole, ivermectin or pyrethroids; antifungal agents such as compounds of the imidazole family such as miconazole, clotrimazole, econazole, ketoconazole, or salts thereof, polyene compounds such as amphotericin B, compounds of the allylamine family such as terbinafine.

In some embodiments, the compositions disclosed herein may comprise additional anti-inflammatory agents, such as steroidal anti-inflammatory agents including hydrocortisone triamcinolone, fluocinonide, betamethasone valerate or clobetasol propionate, or non-steroidal anti-inflammatory agents such as ibuprofen and salts thereof, naproxen and salts thereof, or acetaminophen. In some embodiments, the compositions disclosed herein may comprise anesthetic agents such as the “amide” and “ester” anesthetics such as lidocaine, prilocaine, tetracaine, hydrochloride and derivatives thereof. In some embodiments, the compositions disclosed herein may comprise antipruriginous agents such as thenalidine, trimeprazine, or pramoxine. In some embodiments, the compositions disclosed herein may comprise antiviral agents such as acyclovir. In some embodiments, the compositions disclosed herein may comprise keratolytic agents such as alpha- and beta-hydroxy acids such as glycolic acid or salicylic acid, or urea. In some embodiments, the compositions disclosed herein may comprise anti-free radical agents (antioxidants) such as Vitamin E (alpha tocopherol) and its derivatives, Vitamin C (ascorbic acid), Vitamin A (retinol) and its derivatives, and superoxide dismutases. In some embodiments, the compositions disclosed herein may comprise antiseborrheic agents such as zinc pyrithione and selenium sulfide. In some embodiments, the compositions disclosed herein may comprise antihistamines such as cyproheptadine or hydroxyzine. In some embodiments, the compositions disclosed herein may comprise antipsoriatic agents such as calcipotriene, anthralines, coal tar. In some embodiments, the compositions disclosed herein may comprise immune modulating agents such as imiquimod. In some embodiments, the compositions disclosed herein may comprise calcineurin inhibitors pimecrolimus and tacrolimus.

### Methods for Treating an Inflammatory Skin Condition

Also disclosed herein are methods for treating an inflammatory skin condition. In some embodiments, the compositions and methods disclosed herein are effective for treatment of inflammatory skin conditions such as eczema, dandruff, and acne.

In certain embodiments, the present disclosure provides a method for the treatment or lessening the severity of an inflammatory skin condition such as eczema, dandruff, and acne comprising administering an effective amount of a formulation disclosed herein to a subject in need thereof. In certain embodiments of the present disclosure an “effective amount” of the compound or pharmaceutically acceptable composition is that amount effective for treating or lessening the severity of an inflammatory skin condition, such as eczema, dandruff, and acne. The formulas and compositions according to the methods disclosed herein may be administered using any amount and any topical form of administration effective for treating or lessening the severity of an inflammatory skin condition. The exact amount required will vary from subject to subject, depending on the species, age, and general condition of the subject, the severity of the condition, the particular agent, its mode of administration, and the like. The formulas disclosed herein are, in some embodiments, formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions disclosed herein will be decided by the attending physician within the scope of sound medical judgment. The specific effective dose level for any particular subject or organism will depend upon a variety of factors including the disorder being treated and the severity of the disorder; the activity of the specific compound employed; the specific composition employed; the age, body weight, general health, sex and diet of the subject; the time of administration, topical method of administration; the duration of the treatment; drugs used in combination or coincidental with the specific compound employed, and like factors well known in the medical arts. The term “subject”, as used herein, means an animal, preferably a mammal, and most preferably a human.

Dosage forms for topical administration are disclosed above. Additionally, the present disclosure provides the use of skin patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In certain embodiments, the formulations disclosed herein are applied once daily, twice daily, three times daily, or more. In certain embodiments, the formulations disclosed herein are applied for more than 5 days, more than 10 days, more than 20 days, or more than 30 days. In certain embodiments, the formulations disclosed herein are applied twice daily for about 20 days. According to certain embodiments, the formulations disclosed herein are applied once daily for about 30 days. According to certain embodiments, the formulations disclosed herein are applied once daily for about 35 days. According to certain embodiments, the formulations disclosed herein are applied serially.

In some embodiments, the composition for treating an inflammatory skin condition is administered every hour, every two hours, every three hours, every six hours, every twelve hours, every day, every two days, every three days, every five days, every seven days, every ten days, or every fourteen days.

In some embodiments, the compositions disclosed herein are effective to treat one or more types of inflammatory skin conditions such as rosacea, including erythematotelangiectatic rosacea, papulopustular rosacea, phymatous rosacea, ocular rosacea or combinations thereof. In some embodiments, the compositions disclosed herein are effective to treat the symptoms associated with rosacea, including, for example, papules, pustules, phymas (skin thickening), telangiectasias or erythema associated with rosacea, other skin erythemas, telangiectasias, purpura or the like, and other manifestations associated therewith.

In some embodiments, the compositions disclosed herein are effective to treat other inflammatory conditions of the skin including, but not limited to, keratosis pilaris, lupus miliaris dissemniatus faciei, eczema, dermatitis, such as contact dermatitis, atopic dermatitis, seborrheic dermatitis, nummular dermatitis, generalized exfoliative dermatitis, statis dermatitis, neurodermatitis, lichen simplex chronicus, xerosis and xerotic dermatitis, dyshidrosis and dyshidrotic dermatitis, asteototic dermatitis or other conditions characterized by sensitive skin or a disturbance of the epidermal barrier. In some embodiments, the compositions disclosed herein are effective to treat disorders characterized by rough, dry, cracked or fissured skin, disorders characterized by hyperkeratotic skin such as keratodermas and ichthyosisis and ichthyosiform dermatoses. In some embodiments, the compositions disclosed herein are effective to treat disorders of hair follicles and sebaceous glands, such as acne, perioral dermatitis, and pseudofolliculitis barbae. In some embodiments, the compositions disclosed herein are effective to treat disorders of sweat glands, such as Miliaria, including, but not limited to, Miliaria crystallina, Miliaria rubra, Miliaria profunda, Miliaria pustulosa; sunburn, chronic actinic damage, poikiloderma, radiation dermatitis, actinic purpura (“solar purpura”). In some embodiments, the compositions disclosed herein are effective to treat other inflammatory dermatoses, reactions and conditions of the skin, including, but not limited to, psoriasis, drug eruptions, erythema multiforme, erythema nodosum, and granuloma annulare. In some embodiments, the compositions disclosed herein are effective to treat diseases and conditions characterized by bleeding or bruising such as petechiae, ecchymosis, purpura and the like including any accumulation of blood in the skin due to vascular extravasation, irrespective of size or cause, bleeding or bruising due to any skin injury which may include any trauma including surgical or procedural trauma. In some embodiments, the compositions disclosed herein are effective to treat infection, inflammatory dermatoses or inflammation due to any cause.

In some embodiments, the compositions disclosed herein are effective to treat rosacea, eczema, dermatitis, atopic dermatitis, psoriasis, steroid-responsive dermatoses, pruritis, or xerosis. In some embodiments, the compositions disclosed herein may be used to treat dry, irritated, erythematous or pruriginous skin in subjects with no underlying skin disease, such as, for example, after physical skin trauma or mechanical skin trauma such as shaving (e.g., as a post-shave healer) or tweezing, after bathing, showering, sweating; or after exposure to environmental factors, such as sun, wind, cold temperature, low humidity, hot and humid conditions, radiation, air pollution, smoke or cigarette smoke; or treat said skin irritation or erythema that is as a result of exposure to a topical irritant such as a chemical agent, insect sting or bite, plant exposure, or application of a topically applied drug product, medicament or topical product, such as a fragrance, insect repellant, exfoliant, skin peeling agent, shaving or depilatory preparation, skin or hair cleanser, soap, detergent or conditioner, hair treatment or colorant, antiperspirant, deodorant, sunscreen, tanning agent, moisturizer, astringent, toner, moisturizer, serum, mask, facial or body cosmetic, ointment, cream, lotion, gel, foam, solution, shake, or powder.

In some embodiments, the compositions disclosed herein are effective for treatment of fungal growth on skin, fingernails, and toenails. Without being limited by theory, in some embodiments the carnosine is effective to inhibit fungal growth via imidazole moiety on the histidine residue combined with its hydrophilicity.

In some embodiments, the composition for treating an inflammatory skin condition further comprises at least one constituent selected from the group consisting of an antibiotic agent, a free radical generating agent, an antifungal agent, an antiviral agent, a nucleoside-analog reverse transcriptase inhibitor, a non-nucleoside reverse transcriptase inhibitor, a protease inhibitor, an immunosuppressant, an anti-inflammatory agent, a retinoid agent, a tar agent, an antihistamine agent, and an antipruritic agent.

In some embodiments, the composition for treating an inflammatory skin condition is administered topically.

According to some aspects, the composition for treating an inflammatory skin condition is effective to alter gene expression of human skin cells. As used herein, “human skin cells” are keratinocytes, fibroblasts, melanocytes, Langerhans, and Merkel cells. In some embodiments, the gene expression that is altered by the compositions disclosed herein is selected from one or more of the genes hFLG, hFGF-2, hEGF-1, hCOL1A1, hCOL1A2, hELN, hIGF-1, hTGF-β2, hMMP-2, hFGF-7, and hSDF-1. In some embodiments, one or more of the genes hFLG, hFGF-2, hEGF-1, hCOL1A1, hCOL1A2, hELN, hIGF-1, and hTGF-β2 are upregulated by the compositions disclosed herein. In some embodiments, one or more of the genes hMMP-2, hFGF-7, and hSDF-1 are downregulated by the compositions disclosed herein.

While certain embodiments have been described, these embodiments have been presented by way of example only, and are not intended to limit the scope of the disclosure. Indeed, the novel methods and systems described herein may be embodied in a variety of other forms, Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics or groups described in conjunction, with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover while operations may be depicted in the drawings or described it he specification in particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in certain embodiments, the actual steps taken in the processes illustrated and/or disclosed may differ from those shown in the FIGURES. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods of the present invention. These examples are illustrative only and are not intended to limit the scope of the invention in any way.

### EXAMPLES

### Example 1: Composition for Use in Treatment of Eczema, Dandruff, and Acne

Dandruff Formula: A composition for treatment of eczema was manufactured by first dissolving carnosine in an appropriate volume of distilled water. An appropriate amount of salicylic acid is then added to the carnosine solution to achieve a ratio of carnosine and salicylate of between 1:1 to about 3:1 by weight and stirred at room temperature until reaction is complete and the solution is fully homogenous. In a separate reaction, an appropriate amount of lactic acid is added to the carnosine solution to achieve a ratio of carnosine and lactate of between 1:1 to about 3:1 by weight and stirred at room temperature until reaction is complete and solution is fully homogenous.

Carnitine is also separately dissolved in an appropriate volume of distilled or deionized water. An appropriate amount of glycolic acid is then added to the carnitine solution to achieve a ratio of carnitine and glycolate of between 1:1 to about 3:1 by weight and stirred at room temperature until reaction is complete and solution is fully homogenous.

Each of the formed salts (i.e., carnosine-salicylate, carnosine-lactate, and carnitine-glycolate) is then combined into a single mixture with gentle stirring, followed by the addition of an appropriate amount of hydroxypropyl gamma-cyclodextrin. Trimethyglycine is then added along with 1,3-propanediol, glycerin, and phenoxyethanol with gentle mixing. Then ethylhexylglycerin is added with gentle mixing.

Separately, sodium hyaluronate is mixed with pentylene glycol. The sodium hyaluronate/pentylene glycol mixture is then added to solution containing salts.

In the above steps, appropriate concentrations of ingredients are used to achieve a dandruff formula having the following final concentrations:
Carnosine—4%
Salicylic acid (salicylate)—1.8%
Lactic acid (lactate)—0.8%
Carnitine—1%
Glycolic acid (glycolate)—0.5%
Hydroxypropyl Gamma-cyclodextrin—0.5%
Glycerin—0.5%
1,3 Propanediol—8%
Pentylene glycol—2%
Trimethylglycine—3%
Sodium hyaluronate—0.2%
Phenoxyethanol—0.6%
Ethylhexylglycerin—0.05%

The above formula is a stable and homogenous mixture that is effective for treatment of dandruff in a human subject.

Eczema Formula 1: A second composition for treatment of eczema was manufactured by first dissolving carnosine in an appropriate volume of distilled water. An appropriate amount of lactic acid is then added to the carnosine solution to achieve a ratio of carnosine and lactate of between 1:1 to about 3:1 by weight and stirred at room temperature until reaction is complete and solution is fully homogenous.

Carnitine is also separately dissolved in an appropriate volume of distilled or deionized water. An appropriate amount of lactic acid is then added to the carnitine solution to achieve a ratio of carnitine and lactate of between 1:1 to about 3:1 by weight and stirred at room temperature until reaction is complete and solution is fully homogenous.

Each of the formed salts (i.e., carnosine-lactate, and carnitine-lactate) is then combined into a single mixture with gentle stirring, followed by the addition of an appropriate amount of hydroxypropyl gamma-cyclodextrin. Then allantoin is added to the solution with gentle mixing. Trimethyglycine is then added along with 1,3-propanediol, glycerin, and phenoxyethanol with gentle mixing. Then ethylhexylglycerin is added with gentle stirring.

Separately, sodium hyaluronate is mixed with pentylene glycol. The sodium hyaluronate/pentylene glycol mixture is then added to solution containing salts.

In the above steps, appropriate concentrations of ingredients are used to achieve an eczema formula having the following final concentrations:
Carnosine—2%
Lactic acid (lactate)—1.4%
Carnitine—1%
Hydroxylpropylgamma-cyclodextrin—0.2%
Glycerin—0.5%
1,3-Propanediol—8%
Pentylene glycol—2%
Trimethylglycine—7%
Sodium hyaluronate—0.2%
Phenoxyethanol—0.6%
Ethylhexylglycerin—0.05%
Allantoin—0.5%

The above formula is a stable and homogenous mixture that is effective for treatment of eczema in a human subject.

Eczema Formula 2: A third composition for treatment of eczema was manufactured by first dissolving carnosine in an appropriate volume of distilled water. An appropriate amount of lactic acid is then added to the carnosine solution to achieve a ratio of carnosine and lactate of between 1:1 to about 3:1 by weight and stirred at room temperature until reaction is complete and solution is fully homogenous.

Carnitine is also separately dissolved in an appropriate volume of distilled or deionized water. An appropriate amount of glycolic acid is then added to the carnitine solution to achieve a ratio of carnitine and glycolate of between 1:1 to about 3:1 by weight and stirred at room temperature until reaction is complete and solution is fully homogenous.

Each of the formed salts (i.e., carnosine-lactate, and carnitine-glycolate) is then combined into a single mixture with gentle stirring, followed by the addition of an appropriate amount of hydroxypropyl gamma-cyclodextrin. Then allantoin is added to the solution with gentle mixing. Trimethyglycine is then added along with 1,3-propanediol, glycerin, and phenoxyethanol with gentle mixing. Then ethylhexylglycerin is added with gentle mixing. Separately, sodium hyaluronate is mixed with pentylene glycol. The sodium hyaluronate/pentylene glycol mixture is then added to solution containing salts.

In the above steps, appropriate concentrations of ingredients are used to achieve an eczema formula having the following final concentrations:
Carnosine—5%
Lactic acid (lactate)—2.8%
Carnitine—2%
Glycolic acid (glycolate)—0.3%
Gamma-cyclodextrin—0.5%
Glycerin—0.5%
1,3-Propanediol—8%
Pentylene glycol—2%
Trimethylglycine—7%
Sodium hyaluronate—0.6%
Phenoxyethanol—0.6%
Ethylhexylglycerin—0.05%
Allantoin—0.5%

The above formula is a stable and homogenous mixture that is effective for treatment of eczema in a human subject.

Acne Formula: A composition for treatment of acne was manufactured by first dissolving carnitine in an appropriate volume of distilled water. An appropriate amount of salicylic acid is then added to the carnitine solution to achieve a ratio of carnitine and salicylate of between 1:1 to about 3:1 by weight and stirred at room temperature until reaction is complete and the solution is fully homogenous. In a separate reaction, an appropriate amount of lactic acid is added to the carnitine solution to achieve a ratio of carnitine and lactate of between 1:1 to about 3:1 by weight and stirred at room temperature until reaction is complete and solution is fully homogenous. An appropriate amount of glycolic acid is also added to a separate carnitine solution to achieve a ratio of carnitine and glycolate of between 1:1 to about 3:1 by weight and stirred at room temperature until reaction is complete and solution is fully homogenous.

Each of the formed salts (i.e., carnitine-salicylate, carnitine-lactate, and carnitine-glycolate) is then combined into a single mixture with gentle stirring, followed by the addition of an appropriate amount of hydroxypropyl gamma-cyclodextrin. Trimethyglycine is then added along with 1,3-propanediol, glycerin, and phenoxyethanol with gentle mixing. Then ethylhexylglycerin is added with gentle mixing. Separately, sodium hyaluronate is mixed with pentylene glycol. The sodium hyaluronate/pentylene glycol mixture is then added to solution containing salts.

In the above steps, appropriate concentrations of ingredients are used to achieve an acne treatment formula having the following final concentrations:
Carnosine—2%
Salicylic acid (salicylate)—1.8%
Lactic acid (lactate)—0.8%
Carnitine—1%
Glycolic acid (glycolate)—0.5%
Hydroxypropyl Gamma-cyclodextrin—0.5%
Glycerin—0.5%
1,3 Propanediol—8%
Pentylene glycol—2%
Trimethylglycine—3%
Sodium hyaluronate—0.2%
Phenoxyethanol—0.6%
Ethylhexylglycerin—0.05%

The above formula is a stable and homogenous mixture that is effective for treatment of acne in a human subject.

### Example 2: Failed Formulations

The following formulas are provided as examples that failed to achieve stability and/or homogeneity. The failed formulas generally had one of the following failure modes:
Type 1—amounts of carnosine/carnitine salts and cyclodextrin where salts would not fully go into solution and/or cause salts or cyclodextrin to fall out of solution as a fine crystalline precipitate;
Type 2—at lower ratios of carnosine/carnitine salts to cyclodextrin, where the cyclodextrin is able to solubilize, addition of hyaluronic acid caused an insoluble complex to form and crash out of solution;
Type 3—when the solution is formed of carnosine/carnitine salts and cyclodextrin, the addition of preservation agent (e.g., an aromatic alcohol) along with ethylhexylglycerin form an insoluble complex separates as an oil-like byproduct preventing a homogenous solution.

| Failed Formula 1 | Failure Type |
|---|---|
| Carnosine- 4% | Type 2 |
| Salicylic acid- 1.8% | |
| Lactic acid- 0.8% | |
| Carnitine- 1% | |
| Glycolic acid-0.5% | |
| Hydroxypropyl Gamma-cyclodextrin 1.5% | |
| Glycerin- 0.5% | |
| 1,3 Propanediol- 4% | |
| Pentylene glycol 2% | |
| Trimethylglycine-3% | |
| Sodium hyaluronate- 1% | |
| Phenoxyethanol-0.6% | |
| Ethylhexylglycerin-0.1% | |

| Failed Formula 2 | Failure Type |
|---|---|
| Carnosine- 4% | Type 2 and 3 |
| Salicylic acid- 1.8% | |
| Lactic acid- 0.8% | |
| Carnitine- 1% | |
| Glycolic acid-0.5% | |
| Gamma-cyclodextrin 1.5% | |
| Glycerin- 0.5% | |
| 1,3 Propanediol- 8% | |
| Pentylene glycol 2% | |
| Trimethylglycine-3% | |
| Sodium hyaluronate- 1% | |
| Phenoxyethanol-1% | |
| Ethylhexylglycerin-0.1% | |

| Failed Formula 3 | Failure Type |
|---|---|
| Carnosine- 4% | Type 1 |
| Salicylic acid- 1.8% | |
| Lactic acid- 0.8% | |
| Carnitine- 1% | |
| Glycolic acid-0.5% | |
| Hydroxypropyl Gamma-cyclodextrin 2% | |
| Glycerin- 0.5% | |
| 1,3 Propanediol- 8% | |
| Pentylene glycol 2% | |
| Trimethylglycine-1% | |
| Sodium hyaluronate- 0.2% | |
| Phenoxyethanol-0.6% | |
| Ethylhexylglycerin-0.05% | |

| Failed Formula 4 | Failure Type |
|---|---|
| Carnosine- 4% | Type 3 |
| Salicylic acid- 1.8% | |
| Lactic acid- 0.8% | |
| Carnitine- 1% | |
| Glycolic acid-0.5% | |
| Gamma-cyclodextrin 1.3% | |
| Glycerin- 0.5% | |
| 1,3 Propanediol- 3% | |
| Pentylene glycol 1% | |
| Trimethylglycine-3% | |
| Sodium hyaluronate- 0.4% | |
| Phenoxyethanol-0.9% | |
| Ethylhexylglycerin-0.1% | |

| Failed Formula 5 | Failure Type |
|---|---|
| Carnosine- 4% | Type 2 |
| Salicylic acid- 1.8% | |
| Lactic acid- 0.8% | |
| Carnitine- 1% | |
| Glycolic acid-0.5% | |
| Hydroxypropyl Gamma-cyclodextrin 1.2% | |
| Glycerin- 0.5% | |
| 1,3 Propanediol- 2% | |
| Pentylene glycol 1% | |
| Trimethylglycine-3% | |
| Sodium hyaluronate- 1% | |
| Phenoxyethanol-0.5% | |
| Ethylhexylglycerin-0.1% | |

| Failed Formula 6 | Failure Type |
|---|---|
| Carnosine- 4% | Type 2 |
| Salicylic acid- 1.8% | |
| Lactic acid- 0.8% | |
| Carnitine- 1% | |
| Glycolic acid-0.5% | |
| Gamma-cyclodextrin 1.4% | |
| Glycerin- 0.5% | |
| 1,3 Propanediol- 8% | |
| Pentylene glycol 2% | |
| Trimethylglycine-3% | |
| Sodium hyaluronate- 1% | |
| Phenoxyethanol-0.5% | |
| Ethylhexylglycerin-0.1% | |

| Failed Formula 7 | Failure Type |
|---|---|
| Carnosine- 4% | Type 2 and 3 |
| Salicylic acid- 1.8% | |
| Lactic acid- 0.8% | |
| Carnitine- 1% | |
| Glycolic acid-0.5% | |
| Gamma-cyclodextrin 2% | |
| Glycerin- 1% | |
| 1,3 Propanediol- 8% | |
| Pentylene glycol 2% | |
| Trimethylglycine-5% | |
| Sodium hyaluronate- 0.8% | |
| Phenoxyethanol-0.7% | |
| Ethylhexylglycerin-0.1% | |

| Failed Formula 8 | Failure Type |
|---|---|
| Carnosine- 4% | Type 2 and 3 |
| Salicylic acid- 1.8% | |
| Lactic acid- 0.8% | |
| Carnitine- 1% | |
| Glycolic acid-0.5% | |
| Alpha-cyclodextrin 1.5% | |
| Glycerin- 0.5% | |
| 1,3 Propanediol- 8% | |
| Pentylene glycol 2% | |
| Trimethylglycine-3% | |
| Sodium hyaluronate- 0.2% | |
| Phenoxyethanol-0.6% | |
| Ethylhexylglycerin-0.05% | |

| Failed Formula 9 | Failure Type |
|---|---|
| Carnosine- 4% | Type 2 |
| Salicylic acid- 1.8% | |
| Lactic acid- 0.8% | |
| Carnitine- 1% | |
| Glycolic acid-0.5% | |
| Alpha-cyclodextrin 1.3% | |
| Glycerin- 0.5% | |
| 1,3 Propanediol- 8% | |
| Pentylene glycol 2% | |
| Trimethylglycine-3% | |
| Sodium hyaluronate- 1% | |
| Phenoxyethanol-0.6% | |
| Ethylhexylglycerin-0.05% | |

| Failed Formula 10 | Failure Type |
|---|---|
| Carnosine- 4% | Type 2 and 3 |
| Salicylic acid- 1.8% | |
| Lactic acid- 0.8% | |
| Carnitine- 1% | |
| Glycolic acid-0.5% | |
| Hydroxypropyl beta-cyclodextrin 1.5% | |
| Glycerin- 0.5% | |
| 1,3 Propanediol- 8% | |
| Pentylene glycol 2% | |
| Trimethylglycine-3% | |
| Sodium hyaluronate- 0.8% | |
| Phenoxyethanol-0.8% | |
| Ethylhexylglycerin-0.1% | |

| Failed Formula 11 | Failure Type |
|---|---|
| Carnosine- 4% | Type 2 |
| Salicylic acid- 1.8% | |
| Lactic acid- 0.8% | |
| Carnitine- 1% | |
| Glycolic acid-0.5% | |
| Hydroxypropyl beta-cyclodextrin 1.2% | |
| Glycerin- 0.5% | |
| 1,3 Propanediol- 8% | |
| Pentylene glycol 2% | |
| Trimethylglycine-5% | |
| Sodium hyaluronate- 1% | |
| Phenoxyethanol-0.6% | |
| Ethylhexylglycerin-0.05% | |

### Example 2: Gene Expression Analysis

A stock of Carnosine at 4% (meaning 177 mM) was made from a keratinocyte complete media and Fibroblast complete media at 1:1 ratio. The keratinocyte complete media is composed of Dermal Cell Basal Medium (ATCC, catalog #PCS-200-030) mixed with Keratinocyte Growth Kit (ATCC, catalog #PCS-200-040) as specified by ATCC. The Fibroblast complete media is composed of Eagle's Minimum Essential Medium (EMEM) (ATCC, catalog #30-2003) mixed with 10% of decomplemented fetal bovine serum (Cytiva, catalog #SH30088.03) as specified by ATCC.

Primary adult normal Human Epidermal Keratinocytes (HEKa) (ATCC, catalog #PCS-200-011) were cultivated and expanded as a monoculture in complete Keratinocyte media; epidermal meaning from skin. Adult normal human skin fibroblasts (ATCC, catalog #CRL-2091) were cultivated and expanded as a monoculture in complete Fibroblast media.

When the experiments were set up, at that time only, both cells were cultivated together at a ratio of 1:1, using a made-up media of complete Keratinocyte media and complete Fibroblast media at a ratio 1:1.

The experiments were used to identify biomarkers associated with eczema and dandruff on the co-culture of primary adult normal Epidermal Keratinocytes (HEKa) (ATCC, catalog #PCS-200-011) with adult normal skin fibroblasts (ATCC, catalog #CRL-2091). Both types of cells were plated at the same time and allowed to attach to the plate. After 24h, multiple co-cultures of cells were dosed with different concentrations of Carnosine, each condition in six replicates). After 24 of dosing, the media was discarded and fresh keratinocyte media/fibroblast media (1:1 ratio) without carnosine, was added to each co-culture condition. After an additional 24 h, the cells were lysed using lysis buffer (RLA with beta-mercaptoethanol) from a kit of total RNA extraction (SV Total RNA Isolation System) as specified by the vendor (Promega). Each condition, each replicate was extracted as a unique RNA sample.

After total RNA extraction, RNA was quantified and checked for quality by measuring the Optical Density of each individual RNA sample. A reverse transcription was performed on each individual RNA sample at 300ng of total RNA using the SuperScript™ IV VILO™ Master Mix from vendor ThermoFisher Scientific following manufacturer instructions to produce a cDNA equivalent to each RNA sample in a machine used as a thermocycler.

After reverse transcription, each sample was diluted 1:1 with molecular biology grade water. After dilution of the cDNA samples, real-time PCR was performed on each cDNA sample (15ng) set as multiplex, meaning two Taqmans were used simultaneously for each sample using a real-time PCR machine; one Taqman was the Eukaryotic 18S rRNA Endogenous Control the other Taqman was the target gene. The real-time PCR data is run as comparative Ct (delta delta Ct).

There was a new real-time PCR for each couple Eukaryotic 18S rRNA Endogenous Control the other Taqman was the target gene versus target gene Taqman. All target genes Taqmans were for human genes. Target genes were selected for genes known to play a role in eczema. The different couples were as follows:

Eukaryotic 18S rRNA Endogenous Control Taqman (VIC™/MGB probe, primer limited, ThermoFisher Scientific, catalog #4319413E) and h COL1A2 (ThermoFisher Scientific, Assay ID #HsO1028956_m1)

Eukaryotic 18S rRNA Endogenous Control Taqman (VIC™/MGB probe, primer limited, ThermoFisher Scientific, catalog #4319413E) and h EGF-1 (ThermoFisher Scientific, Assay ID #HsO1099990_m1)

Eukaryotic 18S rRNA Endogenous Control Taqman (VIC™/MGB probe, primer limited, ThermoFisher Scientific, catalog #4319413E) and h ELN (ThermoFisher Scientific, Assay ID #Hs00355783_m1)

Eukaryotic 18S rRNA Endogenous Control Taqman (VIC™/MGB probe, primer limited, ThermoFisher Scientific, catalog #4319413E) and h FLG (ThermoFisher Scientific, Assay ID #Hs00856927_g1)

Eukaryotic 18S rRNA Endogenous Control Taqman (VIC™/MGB probe, primer limited, ThermoFisher Scientific, catalog #4319413E) and h IGF-1 (ThermoFisher Scientific, Assay ID #HsO1547656_m1)

Eukaryotic 18S rRNA Endogenous Control Taqman (VIC™/MGB probe, primer limited, vendor#ThermoFisher Scientific, catalog #4319413E) and h TGF-β-2 (vendor#ThermoFisher Scientific, Assay ID #Hs00234244_m1);

Eukaryotic 18S rRNA Endogenous Control Taqman (VIC™/MGB probe, primer limited, vendor#ThermoFisher Scientific, catalog #4319413E) and h SDF-1 (vendor#ThermoFisher Scientific, Assay ID #Hs03676656_mH)

Eukaryotic 18S rRNA Endogenous Control Taqman (VIC™/MGB probe, primer limited, vendor#ThermoFisher Scientific, catalog #4319413E) and h FGF7, Vendor #ThermoFisher Scientific, Catalog #Hs00940253 ml);

Eukaryotic 18S rRNA Endogenous Control Taqman (VIC™/MGB probe, primer limited, vendor#ThermoFisher Scientific, catalog #4319413E) and h FGF2, Vendor #ThermoFisher Scientific, Catalog #Hs00266645 ml);

Eukaryotic 18S rRNA Endogenous Control Taqman (VIC™/MGB probe, primer limited, vendor#ThermoFisher Scientific, catalog #4319413E) and h MMP-2, Vendor #ThermoFisher Scientific, Catalog #Hs01548727 ml); and

Eukaryotic 18S rRNA Endogenous Control Taqman (VIC™/MGB probe, primer limited, vendor#ThermoFisher Scientific, catalog #4319413E) and h COL1A1, Vendor #ThermoFisher Scientific, Catalog #Hs00164004_m1).

Real-time PCR was analyzed as comparative Ct (delta delta Ct) using the Eukaryotic 18S rRNA Endogenous Control Taqman values as endogenous control, using Excel (Microsoft). t TEST (sample compared to control) was performed for the obtained data, with p value equal of less than 0.05 being considered as specific.

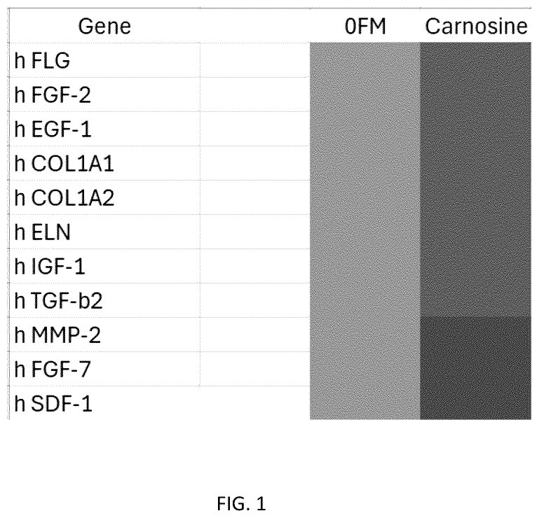

The changes in gene expression observed after treatment are shown in FIG. 1. As shown in FIG. 1, there was no change in gene expression for control cells (“OFM”). In contrast, after carnosine treatment each of hFLG, hFGF-2, hEGF-1, hCOL1A1, hCOL1A2, hELN, hIGF-1, and hTGF-β2 where upregulated, while each of hMMP-2, hFGF-7, and hSDF-1 were downregulated. The genes tested have been found to be involved in each indications pathways from inflammation modulation, enzyme function, tissue repair and regeneration.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

### Various Embodiments

Embodiment 1. A composition that is effective to treat eczema in a subject in need thereof comprising: a solution comprising carnosine, lactic acid, cyclodextrin, carnitine, trimethylglycine, and sodium hyaluronate.

Embodiment 2. The composition of any preceding embodiment, further comprising one or more of allantoin and glycolic acid.

Embodiment 3. The composition of any preceding embodiment, wherein the carnosine and lactic acid form a carnosine lactate salt and the carnitine and lactic acid form a carnitine lactate salt.

Embodiment 4. The composition of any preceding embodiment, wherein the carnosine and glycolic acid form a carnosine glycolate salt and the carnitine and glycolic acid form a carnitine glycolate salt.

Embodiment 5. The composition of any preceding embodiment, wherein the ratio of carnosine lactate and carnitine lactate salts to cyclodextrin is between 1:1 to 3:1 by weight.

Embodiment 6. The composition of any preceding embodiment, wherein the ratio of carnosine glycolate and a carnitine glycolate salts to cyclodextrin is between 1:1 to 3:1 by weight.

Embodiment 7. The composition of any preceding embodiment, wherein the carnosine comprises L-carnosine, D-carnosine, acetyl-carnosine, derivatives thereof, and combinations thereof.

Embodiment 8. The composition of any preceding embodiment, wherein the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

Embodiment 9. The composition of any preceding embodiment, wherein the cyclodextrin is non-crosslinked.

Embodiment 10. The composition of any preceding embodiment, comprising 0.5-6% (wt) carnosine; 0.3-5% (wt) lactic acid; 0.1-1% (wt) hydroxypropyl gamma cyclodextrin; 0.5-3% (wt) carnitine; 3-10% (wt) trimethylglycine; and 0.1-0.8% (wt) sodium hyaluronate.

Embodiment 11. The composition of any preceding embodiment, comprising one or more of 0.4-3.5% (wt) salicylic acid, 0.2-3% (wt) glycolic acid, and 0.5% (wt) allantoin.

Embodiment 12. The composition of any preceding embodiment, comprising 2% (wt) carnosine; 1.4% (wt) lactic acid; 0.2% (wt) hydroxypropyl gamma cyclodextrin; 1% (wt) carnitine; 7% (wt) trimethylglycine; 0.2% (wt) sodium hyaluronate; and 0.5% (wt) allantoin.

Embodiment 13. The composition of any preceding embodiment, comprising 5% (wt) carnosine; 2.8% (wt) lactic acid; 0.3% (wt) glycolic acid; 0.5% (wt) hydroxypropyl gamma cyclodextrin; 2% (wt) carnitine; 7% (wt) trimethylglycine; 0.6% (wt) sodium hyaluronate; and 0.5% (wt) allantoin.

Embodiment 14. The composition of any preceding embodiment, comprising 2% (wt) carnosine; 1.4% (wt) lactic acid; 0.2% (wt) hydroxypropyl gamma cyclodextrin; 1% (wt) carnitine; 7% (wt) trimethylglycine; 0.2% (wt) sodium hyaluronate; 0.5% (wt) allantoin; 0.5% (wt) glycerin; 8% (wt) 1,3-propanediol; 2% (wt) pentylene glycol; 0.6% (wt) phenoxyethanol; 0.05% (wt) ethylhexylglycerine; and 0.5% (wt) allantoin.

Embodiment 15. The composition of any preceding embodiment, comprising 5% (wt) carnosine; 2.8% (wt) lactic acid; 0.3% (wt) glycolic acid; 0.5% (wt) hydroxypropyl gamma cyclodextrin; 2% (wt) carnitine; 7% (wt) trimethylglycine; 0.6% (wt) sodium hyaluronate; 0.5% (wt) glycerin; 8% (wt) 1,3-propanediol; 2% (wt) pentylene glycol; 0.6% (wt) phenoxyethanol; 0.05% (wt) ethylhexylglycerine; and 0.5% (wt) allantoin.

Embodiment 16. The composition of any preceding embodiment, further comprising at least one pharmaceutically acceptable carrier or excipient.

Embodiment 17. The composition of any preceding embodiment, wherein the composition is a patch, a cream, an ointment, a powder, an aerosol spray, or a hydrogel.

Embodiment 18. The composition of any preceding embodiment, wherein the composition is a hydrogel.

Embodiment 19. A method of treating eczema in a subject in need thereof comprising administering to the subject an effective amount of the composition of any preceding embodiment.

Embodiment 20. The method of Embodiment 19, wherein the composition is administered topically.

Embodiment 21. The method of Embodiment 19-20, wherein the composition is administered every hour, every two hours, every three hours, every six hours, every twelve hours, every day, every two days, every three days, every five days, every seven days, every ten days, or every fourteen days.

Embodiment 22. A composition that is effective to treat dandruff in a subject in need thereof comprising: a solution comprising carnosine; salicylic acid; lactic acid; glycolic acid, cyclodextrin; carnitine; trimethylglycine; and sodium hyaluronate.

Embodiment 23. The composition of Embodiment 22, wherein one or more of the carnosine and carnitine react with one or more of salicylic acid, lactic acid, glycolic acid to form a salt.

Embodiment 24. The composition of Embodiment 22-23, wherein the salt is one or more of carnosine salicylate and carnitine glycolate.

Embodiment 25. The composition of Embodiment 22, wherein the carnosine and glycolic acid form a carnosine glycolate salt and the carnitine and glycolic acid form a carnitine glycolate salt.

Embodiment 26. The composition of Embodiment 23, wherein the ratio of carnosine salicylate and carnitine glycolate salts to cyclodextrin is between 1:1 to 3:1 by weight.

Embodiment 27. The composition of Embodiment 23, wherein the ratio of carnosine salicylate and carnitine glycolate salts to cyclodextrin is between 1:1 to 3:1 by weight.

Embodiment 28. The composition of Embodiment 24, wherein the ratio of carnosine salicylate and carnitine glycolate salts to cyclodextrin is between 1:1 to 3:1 by weight.

Embodiment 29. The composition of Embodiment 24, wherein the ratio of carnosine salicylate and carnitine glycolate salts to cyclodextrin is between 1:1 to 3:1 by weight.

Embodiment 30. The composition of Embodiment 25, wherein the ratio of carnosine glycolate and carnitine glycolate salts to cyclodextrin is between 1:1 to 3:1 by weight.

Embodiment 31. The composition of Embodiment 25, wherein the ratio of carnosine glycolate and carnitine glycolate salts to cyclodextrin is between 1:1 to 3:1 by weight.

Embodiment 32. The composition of Embodiment 22, wherein the carnosine comprises L-carnosine, D-carnosine, acetyl-carnosine, derivatives thereof, and combinations thereof.

Embodiment 33. The composition of Embodiment 22, wherein the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

Embodiment 34. The composition of Embodiment 22, wherein the cyclodextrin is non-crosslinked.

Embodiment 35. The composition of Embodiment 22, comprising 0.5-6% (wt) carnosine; 0.4-3.5% (wt) salicylic acid; 0.3-5% (wt) lactic acid; 0.2-3% (wt) glycolic acid, 0.1-1% (wt) cyclodextrin; 0.5-3% (wt) carnitine; 3-10% (wt) trimethylglycine; and 0.1-0.8% (wt) sodium hyaluronate.

Embodiment 36. The composition of Embodiment 22, comprising 4% (wt) carnosine; 1.8% (wt) salicylic acid; 0.8% (wt) lactic acid; 0.5% (wt) glycolic acid, 0.5% (wt) cyclodextrin; 1% (wt) carnitine; 0.5% (wt) glycerin; 8% (wt) 1,3-propanediol; 2% (wt) pentylene glycol; 3% (wt) trimethylglycine; 0.2% (wt) sodium hyaluronate; 0.6% (wt) phenoxyethanol; 0.05% (wt) ethylhexylglycerin.

Embodiment 37. The composition of Embodiment 22, further comprising at least one pharmaceutically acceptable carrier or excipient.

Embodiment 38. The composition of Embodiment 22, wherein the composition is a patch, a cream, an ointment, a powder, an aerosol spray, or a hydrogel.

Embodiment 39. The composition of Embodiment 22, wherein the composition is a hydrogel.

Embodiment 40. A method of treating dandruff in a subject in need thereof comprising administering to the subject an effective amount of the composition of any of Embodiments 22-39.

Embodiment 41. The method of Embodiment 40, wherein the composition is administered topically.

Embodiment 42. The method of Embodiment 40-41, wherein the composition is administered every hour, every two hours, every three hours, every six hours, every twelve hours, every day, every two days, every three days, every five days, every seven days, every ten days, or every fourteen days.

Embodiment 43. A composition that is effective to treat acne comprising: a solution comprising carnosine; salicylic acid; lactic acid; glycolic acid, cyclodextrin; carnitine; trimethylglycine; and sodium hyaluronate.

Embodiment 44. The composition of Embodiment 43, wherein one or more of the carnosine and carnitine react with one or more of salicylic acid, lactic acid, glycolic acid to form a salt.

Embodiment 45. The composition of Embodiment 43-44, wherein the salt is one or more of carnosine salicylate and carnitine glycolate.

Embodiment 46. The composition of Embodiment 43-44, wherein the carnosine and glycolic acid form a carnosine glycolate salt and the carnitine and glycolic acid form a carnitine glycolate salt.

Embodiment 47. The composition of Embodiment 45, wherein the ratio of carnosine salicylate and carnitine glycolate salt to cyclodextrin is between 1:1 to 3:1 by weight.

Embodiment 48. The composition of Embodiment 46, wherein the ratio of carnosine glycolate and carnitine glycolate salts to cyclodextrin is between 1:1 to 3:1 by weight.

Embodiment 49. The composition of Embodiment 44, wherein the ratio of one or more of carnosine salicylate, carnosine glycolate, carnosine lactate, carnitine salicylate, carnitine glycolate, and carnitine lactate salts to cyclodextrin is between 1:1 to 3:1 by weight.

Embodiment 50. The composition of Embodiment 45, wherein the ratio of one or more of carnosine salicylate, carnosine glycolate, and carnosine lactate salts to cyclodextrin is between 1:1 to 3:1 by weight.

Embodiment 51. The composition of Embodiment 46, wherein the ratio of one or more of carnitine salicylate, carnitine glycolate, and carnitine lactate salts to cyclodextrin is between 1:1 to 3:1 by weight.

Embodiment 52. The composition of Embodiment 46, further comprising allantoin.

Embodiment 53. The composition of Embodiment 43, wherein the carnosine comprises L-carnosine, D-carnosine, acetyl-carnosine, derivatives thereof, and combinations thereof.

Embodiment 54. The composition of Embodiment 43, wherein the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

Embodiment 55. The composition of Embodiment 43, wherein the cyclodextrin is non-crosslinked.

Embodiment 56. The composition of Embodiment 43, comprising 0.5-6% (wt) carnosine; 0.4-3.5% (wt) salicylic acid; 0.3-5% (wt) lactic acid; 0.2-3% (wt) glycolic acid, 0.1-1% (wt) cyclodextrin; 0.5-3% (wt) carnitine; 3-10% (wt) trimethylglycine; and 0.1-0.8% (wt) sodium hyaluronate.

Embodiment 57. The composition of Embodiment 43, comprising 0.05% (wt) EDTA, 2% (wt) carnosine, 0.6% (wt) Salicylic acid, 0.45% (wt) lactic acid, 0.5% (wt) glycerin, 0.5% (wt) gamma-cyclodextrin, 2% (wt) pentylene glycol, 8% (wt) propanediol, 0.7% (wt) phenoxyethanol, 0.1% (wt) ethylhexylglycerin, and 0.6% (wt) hyaluronic acid.

Embodiment 58. The composition of Embodiment 43, comprising 0.05% (wt) EDTA, 2% (wt) carnosine, 0.6% (wt) Salicylic acid, 0.45% (wt) lactic acid, 1% (wt) carnitine, 0.44% (wt) glycolic acid, 0.5% (wt) glycerin, 0.5% (wt) gamma-cyclodextrin, 2% (wt) pentylene glycol, 8% (wt) propanediol, 0.7% (wt) phenoxyethanol, 0.05% (wt) ethylhexylglycerin, and 0.6% (wt) hyaluronic acid.

Embodiment 59. The composition of Embodiment 43 comprising, 2% (wt) carnosine, 1.8% (wt) Salicylic acid, 0.8% (wt) lactic acid, 1% (wt) carnitine, 0.5% (wt) glycolic acid, 0.5% (wt) hydroxypropyl gamma-cyclodextrin, 8% (wt) propanediol, 2% (wt) pentylene glycol, 3% (wt) trimethylglycine, 0.2% (wt) sodium hyaluronate, 0.6% (wt) phenoxyethanol, and 0.05% (wt) ethylhexylglycerin.

Embodiment 60. The composition of Embodiment 43, further comprising at least one pharmaceutically acceptable carrier or excipient.

Embodiment 61. The composition of Embodiment 43, wherein the composition is a patch, a cream, an ointment, a powder, an aerosol spray, or a hydrogel.

Embodiment 62. The composition of Embodiment 43, wherein the composition is a hydrogel.

Embodiment 63. A method of treating acne in a subject in need thereof comprising administering to the subject an effective amount of the composition of any of Embodiments 43-62.

Embodiment 64. The method of Embodiment 63, wherein the composition is administered topically.

Embodiment 65. The method of Embodiment 63-64, wherein the composition is administered every hour, every two hours, every three hours, every six hours, every twelve hours, every day, every two days, every three days, every five days, every seven days, every ten days, or every fourteen days.

Embodiment 66. A method for altering gene expression of a human skin cell in a human subject comprising contacting the human skin cell with a composition according to any one of Embodiments 1-18, 22-39, and 43-62; wherein the gene expression that is altered is selected from one or more of the genes hFLG, hFGF-2, hEGF-1, hCOL1A1, hCOL1A2, hELN, hIGF-1, hTGF-β2, hMMP-2, hFGF-7, and hSDF-1.

Embodiment 67. The method of Embodiment 66, wherein one or more of the genes hFLG, hFGF-2, hEGF-1, hCOL1A1, hCOL1A2, hELN, hIGF-1, and hTGF-β2 are upregulated.

Embodiment 68. The method of Embodiment 66, wherein one or more of the genes hMMP-2, hFGF-7, and hSDF-1 are downregulated.

Embodiment 69. A method of making a hydrogel salt composition comprising the steps of: contacting one or more of carnosine and carnitine with an acid to form a carnosine and/or carnitine salt; and combining the carnosine and/or carnitine salt with a cyclodextrin, wherein the ratio of carnosine and/or carnitine salt to cyclodextrin is between 1:1 and 3:1 by weight and wherein the carnosine and/or carnitine salt and cyclodextrin stay in solution.

Embodiment 70. The method of Embodiment 69, wherein the acid is selected from one or more of lactic acid, salicylic acid, glycolic acid, and combinations thereof.

Embodiment 71. The method of Embodiment 70, wherein the carnosine and/or carnitine salt is selected from one or more of carnosine lactate, carnosine salicylate, carnosine glycolate, carnitine lactate, carnitine salicylate, carnitine glycolate, and combinations thereof.

Embodiment 72. The method of Embodiment 69, wherein the cyclodextrin is alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

Embodiment 73. The method of Embodiment 69, wherein the carnosine comprises L-carnosine, D-carnosine, acetyl-carnosine, derivatives thereof, and combinations thereof.

Embodiment 74. The method of Embodiment 69, further comprising the step of adding allantoin to the solution of carnosine and/or carnitine salt and cyclodextrin.

Embodiment 75. The method of Embodiment 74, further comprising the step of adding trimethyglycine 1,3-propanediol, glycerin, and phenoxyethanol to the solution of carnosine and/or carnitine salt, cyclodextrin and allantoin.

Embodiment 76. The method of Embodiment 75, further comprising the step of adding one or more of hyaluronic acid and sodium hyaluronate to the solution of carnosine and/or carnitine salt, cyclodextrin, allantoin, trimethyglycine 1,3-propanediol, glycerin, and phenoxyethanol.

Embodiment 77. The method of Embodiment 76, wherein the hyaluronic acid has a low, medium, or high molecular weight.

Embodiment 78. The method of Embodiment 76, wherein the one or more of hyaluronic acid and sodium hyaluronate is added last to prevent insoluble complexes from forming.

What is claimed:

1. A composition that is effective to treat eczema in a subject in need thereof comprising:
a solution comprising carnosine, lactic acid, cyclodextrin, carnitine, trimethylglycine, and sodium hyaluronate.

2. The composition of claim 1, further comprising one or more of allantoin and glycolic acid.

3. The composition of claim 1, wherein the carnosine and lactic acid form a carnosine lactate salt and the carnitine and lactic acid form a carnitine lactate salt.

4. The composition of claim 2, wherein the carnosine and glycolic acid form a carnosine glycolate salt and the carnitine and glycolic acid form a carnitine glycolate salt.

5. The composition of claim 3, wherein the ratio of carnosine lactate and carnitine lactate salts to cyclodextrin is between 1:1 to 3:1 by weight.

6. The composition of claim 4, wherein the ratio of carnosine glycolate and a carnitine glycolate salts to cyclodextrin is between 1:1 to 3:1 by weight.

7. The composition of claim 1, wherein the carnosine comprises L-carnosine, D-carnosine, acetyl-carnosine, derivatives thereof, and combinations thereof.

8. The composition of claim 1, wherein the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

9. The composition of claim 4, wherein the cyclodextrin is non-crosslinked.

10. The composition of claim 1, comprising 0.5-6% (wt) carnosine; 0.3-5% (wt) lactic acid; 0.1-1% (wt) hydroxypropyl gamma cyclodextrin; 0.5-3% (wt) carnitine; 3-10% (wt) trimethylglycine; and 0.1-0.8% (wt) sodium hyaluronate.

11. The composition of claim 2, comprising one or more of 0.4-3.5% (wt) salicylic acid, 0.2-3% (wt) glycolic acid, and 0.5% (wt) allantoin.

12. The composition of claim 2, comprising 2% (wt) carnosine; 1.4% (wt) lactic acid; 0.2% (wt) hydroxypropyl gamma cyclodextrin; 1% (wt) carnitine; 7% (wt) trimethylglycine; 0.2% (wt) sodium hyaluronate; and 0.5% (wt) allantoin.

13. The composition of claim 2, comprising 5% (wt) carnosine; 2.8% (wt) lactic acid; 0.3% (wt) glycolic acid; 0.5% (wt) hydroxypropyl gamma cyclodextrin; 2% (wt) carnitine; 7% (wt) trimethylglycine; 0.6% (wt) sodium hyaluronate; and 0.5% (wt) allantoin.

14. The composition of claim 2, comprising 2% (wt) carnosine; 1.4% (wt) lactic acid; 0.2% (wt) hydroxypropyl gamma cyclodextrin; 1% (wt) carnitine; 7% (wt) trimethylglycine; 0.2% (wt) sodium hyaluronate; 0.5% (wt) allantoin; 0.5% (wt) glycerin; 8% (wt) 1,3-propanediol; 2% (wt) pentylene glycol; 0.6% (wt) phenoxyethanol; 0.05% (wt) ethylhexylglycerine; and 0.5% (wt) allantoin.

15. The composition of claim 2, comprising 5% (wt) carnosine; 2.8% (wt) lactic acid; 0.3% (wt) glycolic acid; 0.5% (wt) hydroxypropyl gamma cyclodextrin; 2% (wt) carnitine; 7% (wt) trimethylglycine; 0.6% (wt) sodium hyaluronate; 0.5% (wt) glycerin; 8% (wt) 1,3-propanediol; 2% (wt) pentylene glycol; 0.6% (wt) phenoxyethanol; 0.05% (wt) ethylhexylglycerine; and 0.5% (wt) allantoin.

16. The composition of claim 1, further comprising at least one pharmaceutically acceptable carrier or excipient.

17. The composition of claim 1, wherein the composition is a patch, a cream, an ointment, a powder, an aerosol spray, or a hydrogel.

18. The composition of claim 1, wherein the composition is a hydrogel.

19. A method of treating eczema in a subject in need thereof comprising administering to the subject an effective amount of the composition of any one of claims 1-18.

20. The method of claim 19, wherein the composition is administered topically.

21. The method of claim 20, wherein the composition is administered every hour, every two hours, every three hours, every six hours, every twelve hours, every day, every two days, every three days, every five days, every seven days, every ten days, or every fourteen days.

22. A composition that is effective to treat dandruff in a subject in need thereof comprising:
a solution comprising carnosine; salicylic acid; lactic acid; glycolic acid, cyclodextrin; carnitine; trimethylglycine; and sodium hyaluronate.

23. The composition of claim 22, wherein one or more of the carnosine and carnitine react with one or more of salicylic acid, lactic acid, glycolic acid to form a salt.

24. The composition of claim 23, wherein the salt is one or more of carnosine salicylate and carnitine glycolate.

25. The composition of claim 22, wherein the carnosine and glycolic acid form a carnosine glycolate salt and the carnitine and glycolic acid form a carnitine glycolate salt.

26. The composition of claim 23, wherein the ratio of carnosine salicylate and carnitine glycolate salts to cyclodextrin is between 1:1 to 3:1 by weight.

27. The composition of claim 23, wherein the ratio of carnosine salicylate and carnitine glycolate salts to cyclodextrin is between 1:1 to 3:1 by weight.

28. The composition of claim 24, wherein the ratio of carnosine salicylate and carnitine glycolate salts to cyclodextrin is between 1:1 to 3:1 by weight.

29. The composition of claim 24, wherein the ratio of carnosine salicylate and carnitine glycolate salts to cyclodextrin is between 1:1 to 3:1 by weight.

30. The composition of claim 25, wherein the ratio of carnosine glycolate and carnitine glycolate salts to cyclodextrin is between 1:1 to 3:1 by weight.

31. The composition of claim 25, wherein the ratio of carnosine glycolate and carnitine glycolate salts to cyclodextrin is between 1:1 to 3:1 by weight.

32. The composition of claim 22, wherein the carnosine comprises L-carnosine, D-carnosine, acetyl-carnosine, derivatives thereof, and combinations thereof.

33. The composition of claim 22, wherein the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

34. The composition of claim 22, wherein the cyclodextrin is non-crosslinked.

35. The composition of claim 22, comprising 0.5-6% (wt) carnosine; 0.4-3.5% (wt) salicylic acid; 0.3-5% (wt) lactic acid; 0.2-3% (wt) glycolic acid, 0.1-1% (wt) cyclodextrin; 0.5-3% (wt) carnitine; 3-10% (wt) trimethylglycine; and 0.1-0.8% (wt) sodium hyaluronate.

36. The composition of claim 22, comprising 4% (wt) carnosine; 1.8% (wt) salicylic acid; 0.8% (wt) lactic acid; 0.5% (wt) glycolic acid, 0.5% (wt) cyclodextrin; 1% (wt) carnitine; 0.5% (wt) glycerin; 8% (wt) 1,3-propanediol; 2% (wt) pentylene glycol; 3% (wt) trimethylglycine; 0.2% (wt) sodium hyaluronate; 0.6% (wt) phenoxyethanol; 0.05% (wt) ethylhexylglycerin.

37. The composition of claim 22, further comprising at least one pharmaceutically acceptable carrier or excipient.

38. The composition of claim 22, wherein the composition is a patch, a cream, an ointment, a powder, an aerosol spray, or a hydrogel.

39. The composition of claim 22, wherein the composition is a hydrogel.

40. A method of treating dandruff in a subject in need thereof comprising administering to the subject an effective amount of the composition of any of claims 22-39.

41. The method of claim 40, wherein the composition is administered topically.

42. The method of claim 41, wherein the composition is administered every hour, every two hours, every three hours, every six hours, every twelve hours, every day, every two days, every three days, every five days, every seven days, every ten days, or every fourteen days.

43. A composition that is effective to treat acne comprising:
a solution comprising carnosine; salicylic acid; lactic acid; glycolic acid, cyclodextrin; carnitine; trimethylglycine; and sodium hyaluronate.

44. The composition of claim 43, wherein one or more of the carnosine and carnitine react with one or more of salicylic acid, lactic acid, glycolic acid to form a salt.

45. The composition of claim 44, wherein the salt is one or more of carnosine salicylate and carnitine glycolate.

46. The composition of claim 44, wherein the carnosine and glycolic acid form a carnosine glycolate salt and the carnitine and glycolic acid form a carnitine glycolate salt.

47. The composition of claim 45, wherein the ratio of carnosine salicylate and carnitine glycolate salt to cyclodextrin is between 1:1 to 3:1 by weight.

48. The composition of claim 46, wherein the ratio of carnosine glycolate and carnitine glycolate salts to cyclodextrin is between 1:1 to 3:1 by weight.

49. The composition of claim 44, wherein the ratio of one or more of carnosine salicylate, carnosine glycolate, carnosine lactate, carnitine salicylate, carnitine glycolate, and carnitine lactate salts to cyclodextrin is between 1:1 to 3:1 by weight.

50. The composition of claim 45, wherein the ratio of one or more of carnosine salicylate, carnosine glycolate, and carnosine lactate salts to cyclodextrin is between 1:1 to 3:1 by weight.

51. The composition of claim 46, wherein the ratio of one or more of carnitine salicylate, carnitine glycolate, and carnitine lactate salts to cyclodextrin is between 1:1 to 3:1 by weight.

52. The composition of claim 46, further comprising allantoin.

53. The composition of claim 43, wherein the carnosine comprises L-carnosine, D-carnosine, acetyl-carnosine, derivatives thereof, and combinations thereof.

54. The composition of claim 43, wherein the cyclodextrin comprises alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

55. The composition of claim 43, wherein the cyclodextrin is non-crosslinked.

56. The composition of claim 43, comprising 0.5-6% (wt) carnosine; 0.4-3.5% (wt) salicylic acid; 0.3-5% (wt) lactic acid; 0.2-3% (wt) glycolic acid, 0.1-1% (wt) cyclodextrin; 0.5-3% (wt) carnitine; 3-10% (wt) trimethylglycine; and 0.1-0.8% (wt) sodium hyaluronate.

57. The composition of claim 43, comprising 0.05% (wt) EDTA, 2% (wt) carnosine, 0.6% (wt) Salicylic acid, 0.45% (wt) lactic acid, 0.5% (wt) glycerin, 0.5% (wt) gamma-cyclodextrin, 2% (wt) pentylene glycol, 8% (wt) propanediol, 0.7% (wt) phenoxyethanol, 0.1% (wt) ethylhexylglycerin, and 0.6% (wt) hyaluronic acid.

58. The composition of claim 43, comprising 0.05% (wt) EDTA, 2% (wt) carnosine, 0.6% (wt) Salicylic acid, 0.45% (wt) lactic acid, 1% (wt) carnitine, 0.44% (wt) glycolic acid, 0.5% (wt) glycerin, 0.5% (wt) gamma-cyclodextrin, 2% (wt) pentylene glycol, 8% (wt) propanediol, 0.7% (wt) phenoxyethanol, 0.05% (wt) ethylhexylglycerin, and 0.6% (wt) hyaluronic acid.

59. The composition of claim 43 comprising, 2% (wt) carnosine, 1.8% (wt) Salicylic acid, 0.8% (wt) lactic acid, 1% (wt) carnitine, 0.5% (wt) glycolic acid, 0.5% (wt) hydroxypropyl gamma-cyclodextrin, 8% (wt) propanediol, 2% (wt) pentylene glycol, 3% (wt) trimethylglycine, 0.2% (wt) sodium hyaluronate, 0.6% (wt) phenoxyethanol, and 0.05% (wt) ethylhexylglycerin.

60. The composition of claim 43, further comprising at least one pharmaceutically acceptable carrier or excipient.

61. The composition of claim 43, wherein the composition is a patch, a cream, an ointment, a powder, an aerosol spray, or a hydrogel.

62. The composition of claim 43, wherein the composition is a hydrogel.

63. A method of treating acne in a subject in need thereof comprising administering to the subject an effective amount of the composition of any of claims 43-62.

64. The method of claim 63, wherein the composition is administered topically.

65. The method of claim 64, wherein the composition is administered every hour, every two hours, every three hours, every six hours, every twelve hours, every day, every two days, every three days, every five days, every seven days, every ten days, or every fourteen days.

66. A method for altering gene expression of a human skin cell in a human subject comprising contacting the human skin cell with a composition according to any one of claims 1-18, 22-39, and 43-62;
wherein the gene expression that is altered is selected from one or more of the genes hFLG, hFGF-2, hEGF-1, hCOL1A1, hCOL1A2, hELN, hIGF-1, hTGF-β2, hMMP-2, hFGF-7, and hSDF-1.

67. The method of claim 66, wherein one or more of the genes hFLG, hFGF-2, hEGF-1, hCOL1A1, hCOL1A2, hELN, hIGF-1, and hTGF-β2 are upregulated.

68. The method of claim 66, wherein one or more of the genes hMMP-2, hFGF-7, and hSDF-1 are downregulated.

69. A method of making a hydrogel salt composition comprising the steps of:
contacting one or more of carnosine and carnitine with an acid to form a carnosine and/or carnitine salt; and
combining the carnosine and/or carnitine salt with a cyclodextrin, wherein the ratio of carnosine and/or carnitine salt to cyclodextrin is between 1:1 and 3:1 by weight and wherein the carnosine and/or carnitine salt and cyclodextrin stay in solution.

70. The method of claim 69, wherein the acid is selected from one or more of lactic acid, salicylic acid, glycolic acid, and combinations thereof.

71. The method of claim 70, wherein the carnosine and/or carnitine salt is selected from one or more of carnosine lactate, carnosine salicylate, carnosine glycolate, carnitine lactate, carnitine salicylate, carnitine glycolate, and combinations thereof.

72. The method of claim 69, wherein the cyclodextrin is alpha cyclodextrin, beta cyclodextrin, gamma cyclodextrin, hydroxypropyl gamma cyclodextrin, 2-hydroxypropyl-β-cyclodextrin, β-cyclodextrin sulfobutylether, a derivative thereof, or a combination thereof.

73. The method of claim 69, wherein the carnosine comprises L-carnosine, D-carnosine, acetyl-carnosine, derivatives thereof, and combinations thereof.

74. The method of claim 69, further comprising the step of adding allantoin to the solution of carnosine and/or carnitine salt and cyclodextrin.

75. The method of claim 74, further comprising the step of adding trimethyglycine 1,3-propanediol, glycerin, and phenoxyethanol to the solution of carnosine and/or carnitine salt, cyclodextrin and allantoin.

76. The method of claim 75, further comprising the step of adding one or more of hyaluronic acid and sodium hyaluronate to the solution of carnosine and/or carnitine salt, cyclodextrin, allantoin, trimethyglycine 1,3-propanediol, glycerin, and phenoxyethanol.

77. The method of claim 76, wherein the hyaluronic acid has a low, medium, or high molecular weight.

78. The method of claim 76, wherein the one or more of hyaluronic acid and sodium hyaluronate is added last to prevent insoluble complexes from forming.
